(19) United States
(12) Patent Application Publication
(10) Pub. No.: US 2005/0119330 A1
(43) Pub. Date: Jun. 2, 2005

(54) Use of antiproliferative agents in the treatment and prevention of pulmonary proliferative vascular diseases

(76) Inventors: Peter N. Kao, Palo Alto, CA (US); Ronald G. Pearl, Palo Alto, CA (US); Toshihiko Nishimura, Menlo Park, CA (US); John L. Faul, Stanford, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/801,729

(22) Filed: Mar. 15, 2004

Related U.S. Application Data
(60) Provisional application No. 60/455,470, filed on Mar. 17, 2003.

(51) Int. Cl. A61K 31/401; A61K 31/366; A61K 31/255

(52) U.S. Cl. 514/423; 514/460; 514/548

(57) ABSTRACT

Methods of treating lung proliferative vascular disorders by administering an antiproliferative agent are provided. A preferred antiproliferative agent is a HMG-CoA reductase inhibitor, preferably simvastatin. Vascular occlusion in the pulmonary arteries of the patient is reduced as a result of the treatment through a reduction in neointimal hyperplasia and medial hypertrophy, and the restoration of normal endothelial cell function. The treatment also results in a reversal of right side cardiac hypertrophy. Lung proliferative vascular disorders that can be treated include primary pulmonary hypertension, secondary pulmonary hypertension, Eisenmenger's syndrome, chronic thromboembolic disease, pulmonary fibrosis, obliterative bronchiolitis, or lymphangioleiomyomatosis. Dosages and pharmaceutical formulations are provided.

### TECHNICAL FIELD

This invention relates generally to treatment and prevention of disease, particularly pulmonary proliferative vascular diseases, and most particularly, pulmonary hypertension.

### BACKGROUND

Primary pulmonary hypertension (PPH) is a severe disease characterized by progressive obliteration of the lumen of small pulmonary arteries due to inappropriate proliferation of vascular lining endothelial cells. Narrowing of the lumen of small pulmonary arteries increases pulmonary vascular resistance and leads to elevations of pulmonary artery pressures that overload the right ventricle. Untreated, progressive pulmonary arterial hypertension is fatal within three years of diagnosis as a consequence of severe right ventricular congestive heart failure. Sporadic PPH is estimated to occur with an incidence of 3-6 cases per million, however the difficulties of diagnosis suggest that this number significantly underestimates the true prevalence. Sporadic PPH occurs following exposure to anorexigens such as fenfluramine or aminorex, to recreational drugs cocaine or methamphetamine, or in association with liver cirrhosis, scleroderma or systemic lupus erythematosis.

The transforming growth factor (TGF)-β superfamily is a family of circulating proteins that regulate growth and repair of tissue in all organs. Any defect in the signal transduction performed by the TGF-β receptors results in defective antiproliferative signaling, which then results in inappropriate proliferation. One member of the TGF-β superfamily, the bone morphogenetic protein receptor type II (BMPR2), has recently been discovered to be mutated in 55% of cases of familial PPH and 26% of cases of sporadic PPH (Deng, Z. et al. (2000) Am. J. Hum. Genet. 67:734-744; Machado, R. D. et al. (2000) Am. J. Hum. Genet. 68:92-102; Newman, J. H., et al. (2001) New Engl. J. Med. 345:319-324). Many different mutations have been described within the protein-coding exons of the BMPR2/PPH1 gene. These mutations lead to truncations of the receptor protein, or to inactivation of the serine-threonine protein kinase catalytic activity. The receptor operates as a heterodimer, and a haploinsufficiency of the BMPR2 results in defective receptor signaling. The function of the BMPR2 is to transmit signals that cause cells to cease proliferation and to differentiate. Thus, in patients with PPH and mutations in the BMPR2, injury to the pulmonary vascular endothelium, initiated by drugs or infection, leads to an inflammatory and proliferative response that is inadequately suppressed because of defective BMPR2 signaling.

Clinical management of pulmonary hypertension involves treatment of right ventricular congestive heart failure with diuretics and use of digoxin for inotropic benefits. The pulmonary vascular disease is currently treated exclusively with vasodilator agents, such as calcium channel blockers (diltiazem or nifedipine), inhaled nitric oxide (NO), and continuous intravenous infusion of prostacyclin (Flolan). Therapy with intravenous prostacyclin is currently the standard of care for management of PPH. Although prostacyclin frequently produces no acute decreases in pulmonary artery pressures, long-term infusion of prostacyclin can induce partial regression of pulmonary hypertension, and these beneficial effects have been attributed to antiproliferative effects of prostacyclin on the pulmonary vasculature. Therapy with intravenous infusion of prostacyclin is expensive and cumbersome (requiring use of an infusion pump and central venous catheter). Associated morbidities include infections of the indwelling central venous catheter, susceptibility to life-threatening systemic hypotension from drug overdoses, and late development of intractable ascites requiring repeated abdominal paracentesis drainage procedures. Drugs currently in clinical trials for management of PPH are nearly all vasodilators, including prostacyclin analogs that are administered subcutaneously, orally or via inhalation, or oral antagonists of the vasoconstrictor endothelin-1.

Other approaches focus on a possible upregulation of nitric oxide synthase (NOS), present in endothelial cells, since the vascular smooth muscle cells located in veins, arteries, and coronary arteries are relaxed on treatment with NO, a vasodilator (U.S. Pat. Nos. 5,968,983 and 6,425,881 to Kaesemeyer). In an animal study of balloon injuries to carotid arteries, simvastatin treatment showed beneficial effects in inhibiting neointimal proliferation (Indolfi, C. et al. (2000) J. Am. Coll. Cardiol. 35: 214-221). Studies on cultured endothelial cells in vitro have shown that simvastatin increases the expression of endothelial cell nitric oxide synthase, which leads to increased production of nitric oxide (Lauf, U. et al. (1997) J. Biol. Chem. 272: 31725-31729). This result is also described in U.S. Pat. No. 6,147,109 to Liao, where the use of HMG-CoA reductase inhibitors to treat a number of disease conditions, including pulmonary hypertension, is proposed, based on simvastatin effects on endothelial cells in vitro. Effects on mice in vivo involving the upregulation of eNOS were also reported. Based on these results, Liao proposed that statin therapy to increase NOS would provide enhancement of vascular relaxation, and would be useful to treat conditions associated with deficient NOS activity. However, reports concerning the role of NOS in pulmonary vascular diseases are contradictory. Berger et al. stated that it remains uncertain whether impaired endothelium-dependent vasorelaxation is associated with a decrease in NOS activity, and suggested that other alterations in endothelial cell metabolism may be primarily responsible for the impaired vasorelaxation. In short, these authors stated that impaired endothelial-dependent vasorelaxation may occur despite increased NOS activity. (Berger, R, et al. (2001) Am. J. Respir. Crit. Care Med. 163:1493-1499).

Additional investigators have reported that simvastatin exerts direct antiproliferative effects on vascular smooth muscle cells by inhibiting the lipid synthesis of the isoprenoid intermediates farnesylpyrophosphate and geranylgeranylpyrophosphate. These lipid molecules are important in the post-translational modification of Ras and Rho GTP-binding proteins that mediate growth-promoting intracellular signal transduction (Lauf, U. et al. (1999) J. Biol. Chem. 274: 21926-21931). Simvastatin and other lipophilic statins have previously been shown to induce apoptosis in cultured vascular smooth muscle cells through downregulation of bcl-2 and rho prenylation. Statins were shown to induce apoptosis of malignant cells including acute myelogenous leukemia in vitro, and suppressed growth of Lewis lung carcinoma in vivo.

The HMG-CoA reductase inhibitors, statins, improve cardiovascular outcomes independent of their effects on cholesterol reduction (Koh, K. K. (2000) Cardiovascular Res. 47: 648-657); Maron, D. J., et al. (2000) Circulation 101:207-13). Immunosuppressive and anti-inflammatory properties of statins (Kwak, B. et al. (2000) Nat. Med. 6:1399-402; Weitz-Schmidt, G. et al. (2001) Nat. Med. 7:687-92) may contribute to the improved outcome of cardiac transplant patients (Kobashigawa, J. A. et al. (1995) N. Engl. J. Med. 333:621-627), and to the improved survival of patients with atherosclerosis (Maron, D. J. et al. (2000) Circulation 101:207-13; Bustos, C. et al. (1998) J. Am. Coll. Cardiol. 32:2057-2064). Statins can suppress endothelial and vascular smooth muscle cell inflammatory and proliferative responses to injury. These effects involve inhibition of isoprenylation of rho- and rac-family GTPases that couple growth factor receptors to the intracellular MAP/ERK kinase signaling pathways (Laufs, U et al. (1999) J. Biol. Chem. 274:21926-31; Indolfi, C. et al. (2000) J. Am. Coll. Cardiol. 35:214-21). Statins also improve endothelium-dependent relaxation through mechanisms that involve stabilization of endothelial nitric oxide synthase (eNOS) mRNA during hypoxia, and enhancement of Akt/protein kinase B phosphorylation of eNOS leading to increased production of nitric oxide.

To date, the prevention of the development of the symptoms of lung proliferative vascular diseases such as PPH, as well as the reversal of these symptoms, has not been accomplished using vasodilation therapy. Neither the direct action of vasodilators, nor the indirect action of upregulating the gene expression of NOS, provides an effective treatment for PPH. Therefore, there remains a need in the art for more effective therapies to treat and prevent pulmonary hypertensive disease. This need is addressed by the present invention, wherein a method of treatment is provided whereby the vascular occlusion caused by neointimal smooth muscle cell proliferation, which characterizes these diseases, is alleviated.

### SUMMARY OF THE INVENTION

In one embodiment, a method of treating a lung proliferative vascular disorder in a patient is provided, which includes administering an antiproliferative agent. A preferred antiproliferative agent is a HMG-CoA reductase inhibitor. The HMG-CoA reductase inhibitor is present in an amount effective to reduce vascular occlusion in the pulmonary arteries of the patient, but does not substantially increase endothelial cell nitric oxide synthase activity in the endothelial cells of the pulmonary arteries of the patient. The lung proliferative vascular disorder can be primary pulmonary hypertension, secondary pulmonary hypertension, Eisenmenger's syndrome, chronic thromboembolic disease, pulmonary fibrosis, obliterative bronchiolitis, or lymphangioleiomyomatosis. Preferred HMG-CoA reductase inhibitors are lipophilic HMG-CoA reductase inhibitors, such as atorvastatin, cerivastatin, fluvastatin, lovastatin, mevastatin, pravastatin, pitastatin, rosuvastatin and simvastatin.

The HMG-CoA reductase inhibitor is generally administered in a dose of from about 0.1 to about 100 mg/kg per day, and more preferably from about 0.1 to about 20 mg/kg per day.

In other embodiments, the HMG-CoA reductase inhibitor is administered to the patient as a pharmaceutical formulation, optionally including a pharmaceutically acceptable carrier suitable for oral, parenteral, transdermal, transmucosal, or pulmonary delivery.

In a preferred embodiment, a method of treating a patient suffering from a lung proliferative vascular disorder is provided, by administering an antiproliferative agent to the patient. A preferred antiproliferative agent is an HMG-CoA reductase inhibitor, and the lung proliferative vascular disorder is primary pulmonary hypertension, secondary pulmonary hypertension, Eisenmenger's syndrome, chronic thromboembolic disease, pulmonary fibrosis, obliterative bronchiolitis or lymphangioleiomyomatosis.

In other embodiments, an additional active agent can be included with the antiproliferative agent. Suitable additional active agents include, but are not limited to, additional antiproliferative agents, such as macrolide anti-inflammatory agents, diterpenoid triepoxides, geranyl transferase inhibitors, farnesyl transferase inhibitors, endothelin receptor antagonists, and inhibitors of EGF tyrosine kinase receptor signaling, and pharmaceutically acceptable salts and esters thereof.

Other suitable active agents include anticoagulants, vasodilators, and pharmaceutically acceptable salts and esters thereof.

Preferred vasodilators include prostanoids, such as prostacylin, treprostinil, iloprost, beraprost, prostaglandin E₁ or prostaglandin E₂; phosphodiesterase (PDE) inhibitors, such as PDE (V) inhibitors; and nitric oxide, and nitric oxide precursors; and calcium channel blockers, such as nifedipine or diltiazem.

Preferred macrolide anti-inflammatory agents include rapamycin and its derivatives, FK506, erythromycin or azithromycin. A preferred diterpenoid triepoxide is triptolide.

Preferred endothelin receptor antagonists include ambrisentan, BMS207940, bosentan, sitaxsentan or tezosentan.

The lung proliferative vascular disorder is generally characterized by neointimal smooth muscle cell hyperplasia or medial hypertrophy in the pulmonary arteries of the patient. When treated with the antiproliferative agent, the neointimal smooth muscle cell hyperplasia or medial hypertrophy is decreased, thereby reducing the vascular occlusion in the pulmonary arteries of the patient.

Generally, the lung proliferative vascular disorder is further characterized by vascular occlusion in the pulmonary arteries of the patient. The antiproliferative agent induces apoptosis in the neointimal smooth muscle cells, thereby reducing the neointimal smooth muscle cell hyperplasia in the pulmonary arteries of the patient. Upon treatment with the antiproliferative agent, the vascular occlusion is reversed, resulting in a decrease in the resistance to blood flow through the pulmonary arteries. Blood flow is increased by from about 5% to at least about 300%. Similarly, resistance to blood flow is decreased by up to about 3-fold.

The antiproliferative agent can be administered by any conventional means of delivery, generally pulmonary, oral, transmucosal, transdermal and parenteral modes of administration are suitable.

The antiproliferative agent can be administered by inhalation, using a dry powder inhaler, metered dose inhaler, or nebulizer. In a more preferred embodiment, the antiproliferative agent is an HMG-CoA reductase inhibitor, and is co-administered with PGI₂. In an even more preferred embodiment, the HMG-CoA reductase inhibitor is a lipophilic HMG-CoA reductase inhibitor, e.g., simvastatin.

In other embodiment, a method of preventing vascular occlusion in a patient predisposed to developing pulmonary hypertension is provided. Generally, the patient is predisposed to develop pulmonary hypertension due to a genetic defect in the TGF-β superfamily of receptors, for example, due to a haploinsufficiency of bone morphogenetic protein receptor II (BMPR2). Administration of an antiproliferative agent to the patient results in a decrease in the vascular occlusion associated with the development of lung proliferative vascular disease, and a decrease in the resistance to blood flow, thereby alleviating the symptoms of the disease.

In a most preferred embodiment, a method of treating pulmonary hypertension is provided, which includes administration of an antiproliferative agent, and optionally includes administration of additional active agents, including additional antiproliferative agents, such as macrolide anti-inflammatory agents, diterpenoid triepoxides, geranyl transferase inhibitors, farnesyl transferase inhibitors, endothelin receptor antagonists, and inhibitors of EGF tyrosine kinase receptor signaling, and pharmaceutically acceptable salts and esters thereof. Other suitable active agents that can be included are anticoagulants, vasodilators, and pharmaceutically acceptable salts and esters thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and 1B illustrate the effect of simvastatin on the development of pulmonary arterial hypertension in pneumonectomized, monocrotaline-treated rats.

FIG. 2 illustrates the prevention of the development of right ventricular hypertrophy in pneumonectomized, monocrotaline-treated rats.

FIGS. 3A-3E demonstrate that simvastatin attenuates pulmonary artery neointimal formation.

FIG. 4 demonstrates the development of vascular occlusion in pneumonectomized, monocrotaline-treated rats, with and without simvastatin treatment.

FIGS. 5A-5F demonstrates that smooth muscle cells are present in intimal lesions of small pulmonary arteries.

FIGS. 6A-6C illustrates that a decrease in endothelial nitric oxide synthase (eNOS) gene expression occurs during the development of hypertensive pulmonary vascular disease, and that treatment with simvastatin restores gene expression levels toward normal.

FIGS. 7A-7C demonstrate that simvastatin reverses established hypertensive pulmonary vascular disease and right ventricular hypertrophy, and confers 100% survival on rats with existing hypertensive pulmonary vascular disease.

FIGS. 8A-8F demonstrates that simvastatin reverses pulmonary artery medial hypertrophy in muscular pulmonary arteries.

FIGS. 9A-9F demonstrates that simvastatin suppresses proliferation and induces apoptosis of pulmonary artery smooth muscle cells.

FIGS. 10A-10H demonstrates the reversal of neointimal vascular occlusion that occurs when simvastatin is administered, in comparison with the further progression of disease seen when vehicle only is administered.

FIG. 11 illustrates the vascular occlusion in lungs of pneumonectomized, monocrotaline-treated rats treated with vehicle only or with simvastatin for 2, 6 or 13 weeks, and the vascular occlusion in lungs of normal untreated rats (none), and demonstrates that administration of simvastatin causes reversal of vascular occlusion in rats afflicted with hypertensive pulmonary vascular disease.

FIG. 12 illustrates the results of transcriptional profiling of gene expression in neointimal pulmonary vascular disease, and the reversion of gene expression to that observed in normal animals by treatment with simvastatin.

### DETAILED DESCRIPTION OF THE INVENTION

I. Definitions and Nomenclature

Before describing the present invention in detail, it is to be understood that unless otherwise indicated this invention is not limited to specific antiproliferative agents or formulations thereof, as such may vary. It is also to be understood that the terminology used herein is for the purpose of describing particular embodiments only, and is not intended to be limiting. It must be noted that, as used in this specification and the appended claims, the singular forms “a,” “an,” and “the” include plural referents unless the context clearly dictates otherwise. Thus, for example, reference to “an HMG-CoA reductase inhibitor” includes not only a single HMG-CoA reductase inhibitor, but also a combination or mixture of two or more different HMG-CoA reductase inhibitors; reference to “an additional active agent” includes not only a single additional active agent, but also a combination or mixture of two or more additional active agents, and the like.

In describing and claiming the present invention, the following terminology will be used in accordance with the definitions set out below.

The term “vascular occlusion” refers to the decrease in the cross sectional area of the lumen of a blood vessel (an artery or vein) such that blood flow through the blood vessel is obstructed. Generally, the flow of blood is obstructed when there is any decrease in the cross sectional area of lumen of an artery, resulting in a decrease in the quantity of blood that can be carried by the artery, reducing cardiac output.

The term “lung proliferative vascular disorder” refers to any of the proliferative and obliterative vascular disorders affecting the lung. These disorders include primary pulmonary hypertension, secondary pulmonary hypertension, Eisenmenger's syndrome, chronic thromboembolic disease, pulmonary fibrosis, obliterative bronchiolitis, and lymphangioleiomyomatosis. The lung proliferative vascular disorder of particular interest is primary pulmonary hypertension.

The term “antiproliferative agent” refers to an agent having antiproliferative effects on vascular smooth muscle cells and endothelial cells.

The term “patient predisposed to developing pulmonary hypertension” refers to an animal, preferably a mammal, most preferably a human, in which there is a defect in antiproliferative signaling. Defective antiproliferative signaling can be related to defective signaling through the transforming growth factor (TGF)-β superfamily of receptors (Smad). An example of a patient predisposed to developing pulmonary hypertension is a patient having a defect in bone morphogenetic protein 2, resulting in defective BMPR2 signaling, and a deficit of antiproliferative signaling as a result. Other antiproliferative defects are caused, for example, by inadequate control of immune function, resulting in immune-mediated endothelial injury via proinflammatory cytokines (e.g., IL-1), chemokines, or TNF.

The terms “preventing” and “prevention” refer to administration of an agent to decrease the probability of occurrence of a disorder, as well as to reduce the severity of disease symptoms and the worsening of symptoms of existing disease.

The term “effective amount” or “therapeutically effective amount” of an agent as provided herein refers to a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using routine experimentation. Thus, an amount of an antiproliferative agent effective to reduce vascular occlusion in the pulmonary arteries of the patient, for example, is readily determinable using experimental procedures that measure blood flow through the pulmonary arteries, or cardiac output, for example.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” The terms “treatment” and “treating” refer to the administration of an agent, with or without an additional active agent, to a patient in order to decrease symptoms of a disease or disorder.

The terms “condition,” “disease” and “disorder” are used interchangeably herein as referring to a physiological state that can be prevented or treated by administration of a pharmaceutical formulation as described herein, unless explicitly stated otherwise.

The term “nitric oxide synthase” or “NOS” refers to the constitutive form of NOS unless otherwise specified, and not to the inducible form of NOS. The constitutive form of NOS is the form most commonly found active in healthy endothelial cells.

The term “which does not substantially increase endothelial cell nitric oxide synthase activity in the endothelial cells of the pulmonary arteries of the patient” refers to an increase of NOS expression or activity to levels which would exist in normal healthy endothelial tissue, but not to any enhancement of NOS expression or activity above levels that would exist in normal healthy endothelial tissue.

II. Methods of Treating and Preventing Lung Proliferative Vascular Disorders

The present methods are based on the discovery that antiproliferative agents, and HMG-CoA reductase inhibitors, in particular, can be used to prevent or treat the symptoms of pulmonary hypertension and other proliferative vascular disorders of the lung. The antiproliferative agents show efficacy both in (1) preventing the development of smooth muscle cell hyperplasia (including medial hypertrophy), and in (2) inducing apoptosis in diseased and hypertrophied vascular tissues. This discovery is in sharp contrast to earlier ideas postulating that HMG-CoA reductase inhibitors act to increase expression or activity of endothelial cell nitric oxide synthase (NOS), thereby relieving the symptoms of pulmonary hypertension or other vascular disorder by relaxing the vascular smooth muscle cells. Instead, the present applicants show that antiproliferative agents such as HMG-CoA reductase inhibitors are involved in direct resolution of the neointimal smooth muscle hyperplasia and medial hypertrophy that causes the vascular occlusion in disease states associated with lung proliferative vascular disorders. In fact, it is demonstrated herein that antiproliferative agents induce apoptosis of vascular smooth muscle cells, resulting in shrinkage of the tissue and direct resolution of the vascular occlusion. The decrease in vascular occlusion that results is much greater than any vasodilation that could occur from the administration of a vasodilator, even in the presence of normal levels of eNOS.

It was also discovered that administration of the antiproliferative agents causes reversal of the right ventricular hypertrophy associated with pulmonary hypertension and other proliferative vascular disorders of the lung. Finally, it is demonstrated herein that HMG-CoA reductase inhibitors act only to restore expression of eNOS mRNA towards levels seen in normal healthy endothelium in the vascular tissues of the lung, in contrast to prior reports that HMG-CoA reductase inhibitors increase expression of NOS. Antiproliferative agents such as HMG-CoA reductase inhibitors actually decrease the medial hypertrophy and neointimal smooth muscle cell hyperplasia which causes vascular occlusion in lung proliferative vascular disorders, thereby allowing normal endothelial cell growth and function and restoration of the vascular endothelium.

Using both an acute and chronic model for pulmonary hypertension, it is demonstrated herein that antiproliferative agents such as HMG-CoA reductase inhibitors exert potent effects on vascular wall proliferation and inflammation. The HMG-CoA reductase inhibitor, simvastatin, in particular, suppresses the abnormal proliferation of vascular smooth muscle cells, as assessed by reduced PCNA-positive staining in comparison with control animals, and induces apoptosis of pathologic vascular smooth muscle cells, as assessed by increased TUNEL-staining in comparison with control animals. Proliferating vascular smooth muscle cells in pathological neointimal lesions appear to be preferentially susceptible to simvastatin-induced apoptosis.

The acute rat model for pulmonary hypertension involves the following: rats undergo left pneumonectomy so that the entire cardiac output flows through the right lung, and subsequently the rats receive a single subcutaneous injection of the plant alkaloid monocrotaline. The monocrotaline is metabolized to a toxic pyrrole in the liver, and is then released to the venous circulation, where it induces first pass injury to the pulmonary arterial endothelial cells. Treatment with monocrotaline one week after pneumonectomy generates an acute model of pulmonary hypertension, such that five to seven weeks after the monocrotaline injection, the pulmonary vessels showed 90% occlusion by a neointimal proliferation consisting of myofibroblasts. The mean pulmonary artery pressures rose from a normal of 18 mmHg to over 45 mmHg. The right ventricle became hypertrophied and the right ventricle/(left ventricle+septum) (RV/LV&S) ratio increased from 0.28 in normal rats to greater than 0.8 in rats with severe pulmonary arterial hypertension. Death from right ventricular congestive heart failure occurred within 1 to 3 weeks after pulmonary pressures surpass 40 mmHg. By administering the monocrotaline at 4 weeks post pneumonectomy, a chronic model of pulmonary hypertension of more gradual onset is shown.

The acute animal model was previously used to demonstrate that the nonvasoactive immunosuppressants, triptolide and rapamycin, could inhibit the development of pulmonary arterial hypertension and neointimal formation in pneumonectomized, monocrotaline treated rats (Faul, J. L. et al., Am. J. Resp. Crit. Care Med. 162:2252-2258, 2000; Nishimura, T. (2001) Am. J. Resp. Crit. Care Med. 163:498-502). Here, use of the acute animal model demonstrates that the administration of antiproliferative agents such as HMG-CoA reductase inhibitors prevents the development of neointimal smooth muscle hypertrophy and medial hypertrophy that results in vascular occlusion and pulmonary proliferative vascular disease. Further, in a model of chronic pulmonary hypertension, administration of an antiproliferative agent reverses already established neointimal smooth muscle hypertrophy and vascular occlusion. Thus, the present inventors have shown that the administration of an antiproliferative agent can effectively treat established pulmonary proliferative vascular disease.

The rat model for pulmonary hypertension approximates the neointimal proliferation and vascular occlusion by smooth muscle cells that occurs in human pulmonary arterial hypertension. In the rat model, endothelial injury by monocrotaline pyrrole leads to diminished barrier function, allowing extravascular leakage of proteases such as serum elastase and thrombin to act upon extracellular matrix components and vascular cells and trigger an inflammatory response. Similarly, pulmonary arterial hypertension in human patients involves histopathological changes of medial hypertrophy, intimal proliferation and occlusion of microvascular pulmonary arteries, and in severe cases, plexiform arteriopathy. In a comprehensive analysis of 53 lungs from patients with moderate to severe pulmonary arterial hypertension (as a consequence of primary pulmonary hypertension, Eisenmenger's syndrome and chronic thromboembolic disease), most cells within intimal lesions stained positive for a-smooth-muscle actin, and negative for endothelial markers, indicating a myofibroblastic phenotype (Yi, E. S. et al. (2000) Am. J. Respir. Crit. Care Med. 162(4 Pt 1): 1577-86). Only a single layer of cells that lined the vascular lumen stained positively for endothelial markers, CD31 and Factor VIII. In addition, endothelial cells can be transdifferentiated by exposure to TGF-β, and subsequently stain positive with antibodies against Factor VIII related-antigen and α-smooth-muscle actin. Therefore, the histopathological changes observed in human patients of medial hypertrophy, intimal proliferation and occlusion of microvascular pulmonary arteries, and plexiform arteriopathy, were reproduced by the rat model. Therefore, the rat pulmonary hypertension model represents a valid and important model system for understanding the development and progression of small-vessel obliterative, hypertensive pulmonary vascular disease in humans.

Accordingly, in one embodiment, a method of treating a lung proliferative vascular disorder is provided, wherein an antiproliferative agent is administered to the patient. In preferred embodiments, the antiproliferative agent is an HMG-CoA reductase inhibitor. In the most preferred embodiments, the lung proliferative vascular disorder is pulmonary hypertension and the antiproliferative agent is an HMG-CoA reductase inhibitor.

In certain embodiments, the lung proliferative vascular disorder is characterized by neointimal smooth muscle cell hyperplasia in the pulmonary arteries of the patient, and upon treatment with the antiproliferative agent, the neointimal smooth muscle cell hyperplasia is decreased. In some embodiments, the antiproliferative agent induces apoptosis in the neointimal smooth muscle cells, resulting in a reduction in the neointimal smooth muscle cell hyperplasia in the pulmonary arteries of the patient. In other embodiments, the lung proliferative vascular disorder is characterized by medial hypertrophy in the pulmonary arteries of the patient, and treatment with the antiproliferative agent results in a decrease in the medial hypertrophy.

In general, the lung proliferative vascular disorder is characterized by vascular occlusion in the pulmonary arteries of the patient. This vascular occlusion results in pathologically elevated pulmonary vascular resistance. Treatment with the antiproliferative agent causes a reversal in the vascular occlusion, such that the vascular resistance is decreased by up to about 3-fold, and blood flow is increased through the pulmonary arteries. In certain embodiments, the blood flow is increased by at least about 5%. In more preferred embodiments, the blood flow is increased by at least about 300%. In preferred embodiments, the lung proliferative vascular disorder is characterized by pulmonary hypertension, and the hypertension is reversed upon treatment with the antiproliferative agent.

In yet another embodiment, a method of preventing vascular occlusion in a patient predisposed to developing pulmonary hypertension is provided, comprising administering an antiproliferative agent to the patient. Preferably, the patient is predisposed to develop pulmonary hypertension due to a genetic defect in the TGF-β superfamily of receptors of the patient. For example, the patient may have a haploinsufficiency of bone morphogenetic protein receptor II (BMPR2). The haploinsufficiency may predispose the patient to proliferative disorders, and is treatable with an antiproliferative agent, such as an HMG-CoA reductase inhibitor.

In a further embodiment, a method of reversing right ventricular hypertrophy in a patient suffering from pulmonary hypertension is provided. The method comprises administering an antiproliferative agent, preferably an HMG-CoA reductase inhibitor.

In an additional embodiment, a method of treating primary pulmonary hypertension is provided, comprising administering an effective amount of an HMG-CoA reductase inhibitor in an amount effective to reduce vascular occlusion in the lung. However, the HMG-CoA reductase inhibitor does not substantially increase endothelial cell NOS activity in the pulmonary tissues of a patient. Rather, the HMG-CoA reductase inhibitor reverses the vascular occlusion by reversing the neointimal hyperplasia, and promoting the restoration of normal healthy endothelial cells.

In yet other embodiments, pharmaceutical formulations are provided for administration of the antiproliferative agents. In some embodiments, additional active agents and/or antiproliferative agents can be included in the formulations. Additional aspects of the formulations, including dosing and administration are discussed herein, and are within the scope of one skilled in the art.

The determination of an amount of an antiproliferative agent that is effective to reduce vascular occlusion in the pulmonary arteries of the patient, for example, is readily determinable using experimental procedures that measure blood flow through the pulmonary arteries, or cardiac output, for example. Blood flow through the pulmonary arteries can be measured, for example, using right heart catheterization with a Swan-Ganz pulmonary catheter, echo-Doppler ultrasonography of the heart, cardiac MRI or NMR imaging methods, dye dilution techniques and carbon dioxide rebreathing.

A. Antiproliferative Agents

1. HMG-CoA Reductase Inhibitors

HMG-CoA reductase inhibitors, often called “statins,” are in common use for treating dyslipidemia. These drugs are competitive inhibitors of 3-hydroxy-3-methylglutaryl-coenzyme A (HMG-CoA) reductase, which catalyzes an early, rate-limiting step in cholesterol biosynthesis, thereby lowering levels of cholesterol and triglyceride in hyperlipidemic patients. This class of drugs is well known in the art. In addition to their well-known effects on lipid levels, the HMG-CoA reductase inhibitors are also implicated in stabilizing nitric oxide synthase levels, reducing inflammation, stabilizing plaques, and reducing the susceptibility of lipoproteins to oxidation.

Preferred HMG-CoA reductase inhibitors are lipophilic, such as atorvastatin, cerivastatin, compactin, dalvastatin, lovastatin, mevastatin, pitavastatin, pravastatin, rosuvastatin, fluvastatin, and simvastatin. Particularly preferred HMG-CoA reductase inhibitors are atorvastatin, fluvastatin, cerivastatin or simvastatin. Most preferred is the HMG-CoA reductase inhibitor, simvastatin.

2. Additional Antiproliferative Agents

Additional antiproliferative agents include, but are not limited to, macrolide anti-inflammatory agents, diterpenoid triepoxides and endothelin receptor antagonists, inhibitors of EGF tyrosine kinase receptor signaling, and pharmaceutically acceptable salts and esters thereof.

The antiproliferative agent rapamycin and its derivatives are an example of the macrolide anti-inflammatory agents, and are described in Nishimura, T. et al. (2001) Am. J. Respir. Crit. Care Med. 163:498-502, and U.S. Pat. No. 6,384,046 to Schuler and U.S. Pat. No. 6,258,823 to Holt. These compounds are also known to possess immunosuppressant properties. Therefore, in some embodiments, rapamycin derivatives lacking immunosuppressive properties are preferred. In some embodiments, the preferred rapamycin is 40-O-(2-hydroxyethyl)-rapamycin. Other suitable macrolide anti-inflammatory agents include, but are not limited to, erythromycin, azithromycin, and FK506.

Diterpenoid triepoxides, such as triptolide, are described in commonly owned U.S. Pat. No. 6,329,148 to Rosen and Kao, and U.S. Pat. No. 6,294,546 to Rosen, and have been shown to possess antiproliferative properties against tumor cells Compounds of particular interest include triptolide, tripdiolide, triptonide, tripterinin, 16-hydroxytriptolide, triptriolide, and tripchloride; as well as derivatives of triptolide, 16-hydroxytriptolide and tripdiolide (2-hydroxytriptolide) that are derivatized at one or more hydroxyl groups. Such derivatives may be ester derivatives, where the attached ester substituents include one or more amino or carboxylate groups. Prodrugs of particular interest include triptolide succinate sodium salt and triptolide succinate tris(hydroxy-methyl)aminomethane salt.

Endothelin receptor antagonists, include bosentan (Tracleer™), sitaxsentan, ambrisentan (Myogen™), BMS207940 (Bristol Myers Squibb), and tezosentan and are also useful in the present methods. These antagonists and derivatives thereof are described, for example, in U.S. Pat. No. 6,162,927 to Winn and U.S. Pat. No. 6,127,371 to Elliott.

Additional antiproliferative agents that may be useful in the methods described herein include inhibitors of EGF tyrosine kinase receptor signaling, such as, but not limited to, Gleevec (STI571).

Yet other antiproliferative agents that are useful in the present methods are the geranyl and farnesyl transferase inhibitors that interfere with the action of protein:farnesyl transferase and protein:geranylgeranyl transferase, and block ras prenylation, such as described in Cohen, L. et al. (2000) Biochem. Pharmacol. 60:1061-1068. Farnesyl pyrophosphate analogs, such as BMS, Fabre, TR006, TR015, and CAAX peptidomimetics, such as the CAAX tetrapeptide, Cys-Val-Phe-Met, and FTI-276, FTI-277, TR062, A-170634 and L-744,832, are known to inhibit farnesyl transferase (CAAX refers to the amino acid sequence Cys-aliphatic amino acid-aliphatic amino acid-methionine or serine). Protein geranylgeranyl transferase inhibitors include CAAL peptidomimetics (CAAL refers to the amino acid sequence Cys-aliphatic amino acid-aliphatic amino acid-leucine), TR031 and GGTI-298. Preferred farnesyl transferase inhibitors are Zamestra (tipifamib, R115777), SCH66335 and BMS-214662. The radiation sensitizer, L-778,123, is also useful in the present methods.

B. Additional Active Agents

Additional active agents may optionally be used in the present method. Additional active agents that are useful include, but are not limited to anticoagulants, vasodilators, including phosphodiesterase (PDE) inhibitors, and pharmaceutically acceptable salts and esters thereof.

1. Anticoagulants

Anticoagulants that may be used in the methods are known in the art. Preferred anticoagulants are coumadin, warfarin, heparin, heparinoids, and antithrombins.

2. Vasodilators

Vasodilators are compounds that relax vascular smooth muscle and increase blood flow through the affected blood vessel. Vasodilators of particular interest as an adjunct therapy to the present method include prostanoids such as PGI₂ (prostacyclin), PGE₁, PGE₂, treprostinil, Iloprost™, Beraprost™; endothelin receptor antagonists, including but not limited to bosentan, sitaxsentan, ambrisentan (Myogen™, BSF208075); phosphodiesterase (PDE) inhibitors, particularly PDE type V inhibitors such as sildenafil, vardenafil, dipyridamole, ibudilast (T-1032), and pyrazolopyridopyridimidines, such as those described in U.S. Pat. No. 6,300,335 to Campbell.

Nitric oxide, and its metabolic precursors, such as nitroglycerine or arginine, can also be administered in conjunction with the antiproliferative agent, as described herein.

Preferred vasodilators include prostacyclin, which is available in an inhalable formulation under the tradename Iloprost™, or in an oral formulation, under the tradename Beraprost™.

III. Pharmaceutical Formulations and Routes of Administration

The antiproliferative agent may be present in the formulation as a salt, ester, amide, prodrug, or other derivative, or may be functionalized in various ways as will be appreciated by those skilled in the art. Various hydrates of the antiproliferative agents are also included in the formulations of the invention. As is known, one or more water molecules may associate with a particular compound based on, for example, the availability of hydrogen bonding. Methods of producing hydrated species are known and include, for example, placing the active agent in a humid environment. In addition, methods of removing one or more water molecules are known and include, by way of example, lyophilization or exposing the active agent to dry heat.

The invention is not limited with respect to the antiproliferative agents chosen. Lipophilic HMG CoA reductase inhibitors are preferred antiproliferative agents. While not wishing to be bound by theory, it is believed that the more lipophilic derivatives of HMG-CoA reductase inhibitors are more effective due to an enhanced permeability through cell membranes. Furthermore, the formulation is not limited to use of only one HMG-CoA reductase inhibitor, as combinations of HMG-CoA reductase inhibitors or other antiproliferative agents may also be present.

Preferred HMG-CoA reductase inhibitors are atorvastatin, cerivastatin, fluvastatin, lovastatin, mevastatin, pravastatin, pitastatin, rosuvastatin and simvastatin, as well as derivatives thereof, pharmacologically acceptable salts and esters thereof, and combinations of any of the foregoing. More preferred, however, are simvastatin, atorvastatin, cerivastatin, and fluvastatin.

The formulations of the present invention may take any form suitable for delivering the HMG-CoA reductase inhibitor to a patient. Generally, pharmaceutical formulations can be administered orally, by parenteral injection (e.g., intramuscular, intraperitoneal, intravenous or subcutaneous), by transmucosal routes (e.g., rectally, bucally, nasally, vaginally), transdermally or using pulmonary delivery. Pulmonary and oral administration are the preferred modes of administering the HMG-CoA reductase inhibitor to a patient. The formulations are provided in dosage forms appropriate for each route of administration. For example, for pulmonary administration, the formulations may be in the form of a dry powder, aerosol or liquid.

Appropriate dosages for oral formulations are from about 0.1 to about 5 mg per kg body weight. When administered by inhalation, the dose of HMG-CoA reductase inhibitor is administered in a dosage of from about 0.01 to about 10 mg per kg body weight.

A. Oral Formulations

Solid dosage forms for oral administration include capsules, tablets, pills, powders, and granules. In solid dosage forms, the HMG-CoA reductase inhibitor is generally admixed with at least one inert pharmaceutically acceptable carrier such as sucrose, lactose, or starch. Such dosage forms can also comprise, as is normal practice, an additional substance other than an inert diluent, e.g., a lubricating agent such as magnesium stearate. With capsules, tablets, and pills, the dosage forms may also comprise a buffering agent. Tablets and pills can additionally be prepared with enteric coatings, or in controlled release form, using techniques known in the art.

Liquid dosage forms for oral administration include pharmaceutically acceptable emulsions, solutions, suspensions and syrups, with the elixirs containing an inert diluent commonly used in the art, such as water. These compositions can also include one or more adjuvants, such as a wetting agent, an emulsifying agent, a suspending agent, a sweetening agent, a flavoring agent or a perfuming agent.

Preparations for parenteral administration include sterile aqueous or non-aqueous solutions, suspensions, and emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain one or more adjuvants such as a preserving agent, a wetting agent, an emulsifying agent and a dispersing agent. The dosage forms may be sterilized by, for example, filtration through a bacteria-retaining filter, by incorporating sterilizing agents into the compositions, by irradiating the compositions, or by heating the compositions. They can also be manufactured using sterile water, or some other sterile injectable medium, prior to use.

Pharmaceutical formulations for oral or parenteral administration may also comprise a microemulsion containing an antiproliferative agent, and may contain alternative pharmaceutically acceptable carriers, vehicles, additives, etc. particularly suited to oral or parenteral drug administration. Alternatively, a microemulsion containing an antiproliferative agent may be administered orally or parenterally without modification.

Microemulsions are thermodynamically stable, isotropically clear dispersions of two immiscible liquids, such as oil and water, stabilized by an interfacial film of surfactant molecules (Encyclopedia of Pharmaceutical Technology (New York: Marcel Dekker, 1992), volume 9). For the preparation of microemulsions, surfactant (emulsifier), co-surfactant (co-emulsifier), an oil phase and a water phase are necessary. Suitable surfactants include any surfactants that are useful in the preparation of emulsions, e.g., emulsifiers that are typically used in the preparation of creams. The co-surfactant (or “co-emulsifer”) is generally selected from the group of polyglycerol derivatives, glycerol derivatives and fatty alcohols. Preferred emulsifier/co-emulsifier combinations are generally although not necessarily selected from the group consisting of: glyceryl monostearate and polyoxyethylene stearate; polyethylene glycol and ethylene glycol palmitostearate; and caprilic and capric triglycerides and oleoyl macrogolglycerides. The water phase includes not only water but also, typically, buffers, glucose, propylene glycol, polyethylene glycols, preferably lower molecular weight polyethylene glycols (e.g., PEG 300 and PEG 400), and/or glycerol, and the like, while the oil phase will generally comprise, for example, fatty acid esters, modified vegetable oils, silicone oils, mixtures of mono- di- and triglycerides, mono- and di-esters of PEG (e.g., oleoyl macrogol glycerides), etc.

B. Dry Powder Formulations

The dry powder formulations as described herein include, at a minimum, an antiproliferative agent, preferably an HMG-CoA reductase inhibitor. Such dry powder formulations can be administered via pulmonary inhalation to a patient without the benefit of a carrier. Preferably, dry powders formulations that do not include a carrier are administered with the aid of, for example, a dry powder inhaler as described in section IV, infra.

Preferably, however, the dry powder formulations described herein include one or more pharmaceutically acceptable carriers. Although any carrier suitable for pulmonary drug administration may be used, pharmaceutical sugars are particularly preferred for use as carriers in the present invention. Preferred pharmaceutical sugars include those selected from the group consisting of fructose, galactose, glucose, lactitol, lactose, maltitol, maltose, mannitol, melezitose, myoinositol, palatinite, raffinose, stachyose, sucrose, trehalose, xylitol, hydrates thereof, and combinations of any of the foregoing. It is particularly preferred, however, that lactose, e.g., lactose U.S.P., serves as the carrier in the present invention when the formulation is a dry powder.

Once selected, the antiproliferative agent, preferably an HMG-CoA reductase inhibitor, or antiproliferative agents in combination, is blended to form a substantially homogeneous powder mixture. Techniques involved with the preparation of such powders are well known in the art. Briefly stated, however, the preparation generally includes the steps of reducing the particle size of each active agent (again, alone or in combination), and blending. Of course, reducing the particle size of each active agent is not required when a commercially available product having a suitable particle size is used. Techniques for reducing the particle size include, for example, using mills such as an air-jet mill or a ball mill. The active agents should have a particle size diameter of between about 0.1 μm to about 65 μm for pulmonary administration. It is preferred that the active agent particles are about 1 μm to about 10 μm, more preferably about 2 μm to about 5 μm in diameter.

Similarly, the particle size of the remaining components, e.g., carrier, excipient, etc., must be controlled as well. The same techniques described above for reducing the particle size of the antiproliferative agent may be used to reduce the particle size of the remaining components. Again, such techniques are not required when the component is available commercially in the desired particle size range. Preferably, the remaining components, particularly the carrier, have a particle size from about 30 μm to about 100 μm in diameter, with sizes from about 30 μm to about 70 μm most preferred.

For any given particle size range, it is preferred that at least about 60%, more preferably at least about 70%, still more preferably at least about 85%, of the stated particles have a size within the stated or given range. It is most preferred, however that at least about 90% of the particles have the size in the stated or given range. For example, when a component is stated to have a particle size less than 10 μm, it is most preferred that at least 90% of the particles of that component have a particle size of less than 10 μm.

As previously stated, some components of the formulation may be commercially available in the desired particle size range. For example, a preferred lactose product for use in some embodiments of the present invention is the PHARMATOSE™ 325 brand of lactose monohydrate available from DMV International, Veghel, The Netherlands. According to the manufacturer, 100% of the lactose particles have a particle size of less than 100 μm, and only 5 to 10% of the particles have a particle size of less than 32 μm. Furthermore, a minimum of 70% of the lactose particles are stated to have a particle size of less than 63 μm. Advantageously, particle size manipulation steps are avoided when components are commercially available in the desired particle size range.

Preferably, the particle size reduction of the antiproliferative agent and the particle size reduction of the remaining components are carried out separately. In this way, it is possible to provide a formulation in which the particle size of the antiproliferative agent is smaller than the particle size of, for example, the carrier. The advantage of such a formulation is that the antiproliferative agent can penetrate deeply into the pulmonary tract while the carrier (having a relatively larger particle size) is retained in the upper airways.

Conventional blending techniques known to those skilled in the art may be used for combining antiproliferative agents with the carrier and/or remaining components. Such blending techniques include passing the combined powders through a sifter or blending, for example, the active agents and carrier in a powder blender such as a “double cone” blender or a “V-blender.” No matter which technique is employed, however, it is necessary that the resulting powder is a substantially homogeneous mixture. Typically, the antiproliferative agent, preferably an HMG-CoA reductase inhibitor will make up from about 0.01% to about 99% of the total formulation, preferably from about 0.05% to 50% of the total formulation by weight.

After blending, the powder formulation may, if desired, be portioned and/or otherwise processed into unit dose quantities, e.g., portioned into unit dose quantities and individually placed within a dosage form or drug delivery system. Alternatively, the powder formulation may be loaded into a dosage form or drug delivery device and not “metered out” into unit doses until used. Although any dosage form that contains a unit dose of the formulation is acceptable, capsules are preferred. The capsule material may be either hard or soft, and, as will be appreciated by those skilled in the art, typically comprises a water-soluble compound such as gelatin, starch or a cellulosic material. Preferably, the capsules are composed of a cellulosic material, e.g., hydroxypropyl methylcellulose (HPMC). The capsules may be sealed, such as with gelatin bands or the like. See, for example, Remington: The Science and Practice of Pharmacy, Twentieth Edition (Easton, Pa.: Mack Publishing Co., 2000), which describes materials and methods for preparing encapsulated pharmaceuticals. Thus, each capsule or dosage form will typically contain a therapeutically effective dose of each active agent.

Alternatively, the dosage forms may contain less than a therapeutically effective dose in which case administration of two or more dosage forms would be required to achieve the therapeutically effective dose.

C. Aerosol Formulations

The formulations of the present invention may also take the form of an aerosol composition for inhalation. Aerosol formulations are known to those skilled in the art and are described in Remington: The Science and Practice of Pharmacy, supra. Briefly, the aerosol formulation of the invention is either a solution aerosol in which the antiproliferative agent is soluble in the carrier (e.g., propellant) and optional solvent or a dispersion aerosol in which the antiproliferative agent is suspended or dispersed throughout the carrier and optional solvent. It is preferred that the aerosol formulations of the invention are in the form of a dispersion aerosol.

The carrier in the aerosol formulations of the invention is generally a propellant, usually a compressed gas, e.g., air, nitrogen, nitrous oxide, and CO₂, a mixture of compressed gases, a liquefied gas or a mixture of liquefied gases. A mixture of propellants, when present in the formulations, may be comprised of two, three, four or propellants. Preferred mixtures of propellants, however, comprise only two propellants. Any propellant used in the art of preparing aerosol formulations may be used.

Typically, the propellant is a chlorofluorocarbon, a hydrochlorofluorocarbon, a hydrogen-containing fluorocarbon, a perfluorocarbon, a hydrocarbon or a mixture thereof. Preferably, the propellant is a hydrochlorofluorocarbon, a hydrogen-containing fluorocarbon, a perfluorocarbon or a mixture thereof.

Preferred chlorofluorocarbons include dichlorotetrafluoroethane (e.g., CClF₂CClF₂ and CCl₂FCF₃), trichloromonofluoromethane, dichlorodifluoromethane, chloropentafluoroethane, and mixtures thereof. Preferred hydrochlorofluorocarbons include monochlorodifluoromethane, monochlorodifluoroethane (e.g., 1-chloro-1,1-difluoroethane), and mixtures thereof. Preferred hydrogen-containing fluorocarbons include C₁₋₄ hydrogen-containing fluorocarbons such as CHF₂CHF₂, 1,1,1,2-tetrafluoroethane (HFA-134a), difluoroethane (e.g., 1,1-difluoroethane), 1,1,1,2,3,3,3-heptafluoropropane (HFA-227), and mixtures thereof. Preferred perfluorocarbons include CF₃CF₃, CF₃CF₂CF₃, octafluorocyclobutane, and mixtures thereof. Preferred hydrocarbons include propane, isobutane, n-butane, dimethyl ether, and mixtures thereof. Most preferably, the propellant is selected from the group consisting of difluoroethane, CHF₂CHF₂, 1,1,1,2-tetrafluoroethane, 1,1,1,2,3,3,3-heptafluoropropane, CF₃CF₃, CF₃CF₂CF₃, octafluorocyclobutane, and mixtures of any of the foregoing.

As will be appreciated by one skilled in the art, the aerosol formulations of the invention may include one or more excipients. For example, the aerosol formulations may contain: a solvent (e.g., water, ethanol and mixtures thereof) for increasing the solubility of the antiproliferative agent; an antioxidant (e.g., ascorbic acid) for inhibiting oxidative degradation of the active agents; a dispersing agent (e.g., sorbitan trioleate, oleyl alcohol, oleic acid, lecithin, e.g., soya lecithin, corn oil, or combinations thereof) for preventing agglomeration of particles; and/or a lubricant (e.g., isopropyl myristate) for providing slippage between particles and lubricating the components, e.g., the valve and spring, of the inhaler.

As described with respect to dry powder formulations in Section B, the particle size released from aerosol formulations must be appropriate for pulmonary administration. Solution aerosols inherently produce small particles upon actuation of the inhaler given that the antiproliferative agent is expelled along with the carrier, i.e., propellant, solution as it evaporates. Consequently, solution aerosols produce sufficiently small particles, e.g., within a range of about 0.1 μm to about 65 μm, of antiproliferative agent upon administration. In contrast, dispersion aerosols contain undissolved antiproliferative agent in which particle size remains constant, i.e., the size of the particles in the dispersion aerosol remains unchanged as the antiproliferative agent is delivered to the patient. Thus, the antiproliferative agent must have an appropriate particle size before being formulated into a dispersion aerosol. Consequently, methods of reducing the particle size of the antiproliferative agent for the dry powder formulations described above are equally applicable for preparing active agents with an appropriate particle size in a dispersion aerosol. Furthermore, the same ranges of particle sizes preferred for the dry powder formulations are equally applicable for dispersion aerosols.

The aerosol formulation may be prepared by employing a cold filling process. Initially, the components of the aerosol formulation and an aerosol container are cooled, e.g., to about −40° C., such that the carrier, i.e., propellant, is a liquid. All components except for the carrier are placed into the aerosol container. Thereafter, the carrier is added, the components mixed, and a valve assembly inserted into place. The valve assembly is then crimped such that the container is airtight. Thereafter, the container and formulation contained therein are allowed to return to ambient temperature.

As an alternative to the cold filling process, the aerosol formulation may be prepared by transfer of a carrier from a bulk container. In such a process, the components except for the carrier are initially placed into an empty aerosol container. A valve assembly is then inserted and crimped into place. The carrier, under pressure and in liquid form, is metered through the valve assembly from a bulk container or tank of carrier. The container housing the formulation is checked to ensure that the pressurized contents do not leak.

For both of these methods of preparing the aerosol formulations, the antiproliferative agent, preferably an HMG-CoA reductase inhibitor, generally represents from about 0.1 wt. % to about 40 wt. % of the total formulation. It is preferred, however, that the HMG-CoA reductase inhibitor represent about 2 wt. % to about 20 wt. % of the total formulation, with 5 wt. % to about 15 wt. % being most preferred.

D. Liquid Formulations

The formulations of the present invention may also take the form of a liquid composition for inhalation. Liquid formulations are well known in the art. See, for example, Remington: The Science and Practice of Pharmacy, supra. It is preferred that the liquid is an aqueous suspension, although aqueous solutions may be used as well. The liquid formulations include one or more carriers in addition to the antiproliferative agent. Generally, the carrier is a sodium chloride solution having a concentration such that the formulation is isotonic relative to normal body fluid. In addition to the carrier, the liquid formulations may contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Combining the components followed by conventional mixing results in a liquid formulation suitable for inhalation. Typically, the antiproliferative agent will be an HMG-CoA Reductase Inhibitor, and will make up from about 0.01% to about 40% of the total formulation.

IV. Utility and Administration

The invention provides a method for treating a patient suffering from or prone to a condition characterized by vascular occlusion, particularly in the lung, comprising administering to the patient a pharmaceutical formulation comprising an antiproliferative agent, preferably an HMG-CoA reductase inhibitor. In one preferred embodiment, the formulation is administered via inhalation for pulmonary drug administration. In another preferred embodiment, the formulation is administered orally. The formulation comprises: an antiproliferative agent, preferably an HMG-CoA reductase inhibitor, and pharmacologically acceptable salts, esters and derivatives thereof; and optionally, one or more additional active agent. A pharmaceutically acceptable carrier suitable for pulmonary drug administration can also be included. When administered via inhalation, bronchodilators such as selective β2 adrenergic agonists, e.g., albuterol, metaproterenol, salmeterol and terbutaline, as well as theophylline, milrinone and amrinone, can also be included in the formulation or can be used as a pretreatment, to ensure thorough penetration of the formulation throughout the lung.

Additional advantages result when the formulations are administered via the preferred dry powder inhalers described in section IV, infra. Such dry powder inhalers assure that patients, particularly those patients that have traditionally had trouble using inhalers such as children or the elderly, obtain the complete dose. Even medical personnel who are responsible for monitoring and instructing patients in optimal inhaler use lack the rudimentary skills associated with MDIs. See Hanania et al. (1994) Chest 105(1):111-116. Administration of the complete dose is ensured with these preferred dry powder inhalers since little effort in inhalation is required in order to deliver all of the dose to the lungs. This is in contrast to, for example, metered-dose inhalers with which patients must coordinate the actuation of the inhaler with a deep and prolonged inhalation to ensure that the entire dose is received. As a result of the foregoing advantages, the dry powder inhalers described herein may be efficient in delivering the present formulations in reduced dosages, i.e., 5% to 15% less than the dose used in conventional devices.

The actual amount of each active agent in the formulation will, of course, depend upon the age, weight, and general condition of the subject, the severity of the condition being treated, and the judgment of the prescribing physician. Therapeutically effective amounts are known to those skilled in the art and/or are described in the pertinent reference texts and literature. An effective amount of the formulation may be administered with a single administration, e.g., serially administration of the contents of a single capsule containing a therapeutically effective amount of the formulation via a dry powder inhaler or a single actuation of an aerosol inhaler designed to deliver a therapeutically effective amount of the formulation. Alternatively, a patient can obtain an effective amount of the formulation by, for example, administering multiple doses, e.g., serially administering the contents of multiple capsules containing the formulation via a dry powder inhaler.

The formulations of the invention may be administered via oral or nasal inhalation. For oral administration, the patient inhales the formulation through the mouth. The inhaled formulation progressively comes into contact with the air passages of the mouth and throat area, the upper respiratory tract, e.g., trachea, and finally the lower respiratory tract, e.g., bronchioles.

The antiproliferative agent is then absorbed through the tracheal branches and the alveoli and comes into contact with the lung arterioles, where it acts to prevent the progression and reverse existing neointimal hypertrophy, thereby facilitating blood flow through the pulmonary vascular bed.

Nasal inhalation is similar to oral inhalation except that the patient inhales the formulation through the nares, preferably one at a time. For example, the formulation may be administered via a pump spray in which the patient administers a spray in the left nare followed by administration in the right nare.

V. Inhalers

The invention also provides a dry powder inhaler containing a formulation as described herein. Dry powder inhalers are well known to those skilled in the art. Preferably, the dry powder inhaler includes at least one capsule (preferably a hydroxypropyl methylcellulose capsule) containing a unit dose of the formulation. The patient self-administers the dose by inhaling (via oral or nasal inhalation) the dry powder formulation from the inhaler. In this manner, delivery of the dry powder formulation to the pulmonary system is effected. One example of a dry powder inhaler is described in U.S. Pat. No. 5,673,686 to Villax et al. and U.S. Pat. No. 5,881,721 to Bunce et al.

Dry powder formulations can also be administered using a metered dose inhaler or “MDI.” A MDI generally is a unit comprising a can, a crimped cap covering the mouth of the can, and a drug metering valve situated in the cap. A “MDI system” also includes a suitable channeling device. The term “drug metering valve” or “MDI valve” refers to a valve and its associated mechanisms that delivers a predetermined amount of drug formulation from an MDI upon each activation. The channeling device may comprise, for example, an actuating device for the valve and a cylindrical or cone-like passage through which medicament may be delivered from the filled MDI can via the MDI valve to the nose or mouth of a patient, e.g. a mouthpiece actuator. The relation of the parts of a typical MDI is illustrated in U.S. Pat. No. 5,261,538 to Evans.

Most often the MDI can and cap are made of aluminum or an alloy of aluminum, although other metals not affected by the drug formulation, such as stainless steel, an alloy of copper, or tin plate, may be used. An MDI can may also be fabricated from glass or plastic. Preferably, however, the MDI cans employed in the present methods are made of aluminium or an alloy thereof. Advantageously, strengthened aluminium or aluminum alloy MDI cans may be employed. Such strengthened MDI cans are capable of withstanding particularly stressful coating and curing conditions, e.g. particularly high temperatures, which may be required for certain fluorocarbon polymers. Strengthened MDI cans which have a reduced tendency to malform under high temperatures include MDI cans comprising side walls and a base of increased thickness and MDI cans comprising a substantially ellipsoidal base (which increases the angle between the side walls and the base of the can), rather than the hemispherical base of standard MDI cans. MDI cans having an ellipsoidal base offer the further advantage of facilitating the coating process.

The drug metering valve consists of parts usually made of stainless steel, a pharmacologically inert and propellant resistant polymer, such as acetal, polyamide (e.g., Nylon®), polycarbonate, polyester, fluorocarbon polymer (e.g., Teflon®) or a combination of these materials. Additionally, seals and “O” rings of various materials (e.g., nitrile rubbers, polyurethane, acetyl resin, fluorocarbon polymers), or other elastomeric materials are employed in and around the valve.

Aerosol formulations can be generated using nebulizers, described in U.S. Pat. No. 6,387,886 to Montgomery, and nebulize an aqueous formulation into aerosolized droplets of a size predominantly in the range from 1-5 μm. Preferably at least 70% and more preferably greater than 90% of all generated aerosol particles are within 1-5 μm size range. Two types of nebulizers, jet and ultrasonic, are currently available that can produce and deliver particles between the 1 and 5 μm particle size that is optimal for pulmonary delivery. The jet nebulizer works by air pressure to break a liquid solution into aerosolized droplets. The ultrasonic nebulizer utilizes a piezoelectric crystal that shears a liquid into small aerosolized droplets. However, both devices are sensitive to the pH and ionic strength of the formulation. The optimal buffering and osmotic additives can readily be determined by one of skill in the art of preparing aerosolizable formulations.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

### EXPERIMENTAL

The practice of the present invention will employ, unless otherwise indicated, conventional techniques of pharmaceutical formulation and the like, which are within the skill of the art. Such techniques are explained fully in the literature.

In the following examples, efforts have been made to ensure accuracy with respect to numbers used (e.g., amounts, temperature, etc.) but some experimental error and deviation should be accounted for. Unless indicated otherwise, temperature is in degrees Celsius and pressure is at or near atmospheric. All reagents were obtained commercially unless otherwise indicated.

The following abbreviations are also used:
MPAP: mean pulmonary arterial pressure
RVSP: right ventricular systolic pressures
RV/LV+S: the weight ratio of (right ventricle)/(left ventricle and septum)
VOS: vascular occlusion score
eNOS: endothelial cell Nitric Oxide Synthase
H&E stain: Hematoxylin and Eosin stain

### Example 1

### Pulmonary Arterial Hypertension in an Acute Model

Methods: On Day, 0, thirty pathogen-free 13-week-old, male Sprague-Dawley rats (350-400 g) were intubated, anesthetized with halothane (0.5%), and then underwent left pneumonectomy via thoracotomy as described in Faul, J. L. et al. (2000) Am. J. Respir. Crit. Care Med. 162:2252-2258; and Nishimura, R. et al., (2001) Am. J. Respir. Crit. Care Med. 163:498-502. On Day 7, rats received a single subcutaneous injection in the right hind limb of the plant alkaloid monocrotaline (60 mg/kg; Sigma, St. Louis, Mo.). Rats that were not subjected to any treatment were also included in the study (“normal” or “N”).

Twenty-four rats received monocrotaline (60 mg/kg s.c.), seven days after pneumonectomy. The rats were separated into four treatment groups: rats treated with simvastatin on days 5 through 35 (whole treatment or “PMS₅₋₃₅”); rats treated with simvastatin on days 5 through 14 only (early treatment, or “PMS₅₋₁₄”); rats treated with simvastatin on days 15 through 35 only (late treatment, or “PMS₁₄₋₃₅”); and rats given no simvastatin treatment (administered only vehicle, or “PMV”). Rats that were not subjected to any treatment were also included in the study (“normal” or “N”). Rats in the control group, PMV (n=6), received vehicle (V) by oral gavage every day, beginning on Day 5. Rats in treatment group PMS₅₋₃₅ received simvastatin (40 mg/kg/day) by oral gavage on Days 5-35. Rats in treatment group PMS₅₋₁₄ received simvastatin (2 mg/kg/day) by oral gavage from Days 5 to 14. Rats in treatment group PMS₁₅₋₃₅ received simvastatin by oral gavage (40 mg/kg/day) from Days 15 to 35.

On Day 35, rats underwent pulmonary artery catheterization for measurements of mean pulmonary artery pressures and right ventricular systolic pressures. Mean arterial, mean pulmonary arterial and right ventricular systolic blood pressures (mPAP and RVSP, respectively) were measured as described by Faul et al. and Nishimura et al. After exsanguination, the right lung, right ventricle and left ventricle plus septum were collected for histology. The organs were weighed and preserved and the RV/LV&S ratios determined. Organs fixed in formalin were embedded in paraffin, cut in 3 micron sections and stained with hematoxylin and eosin, and with elastin-von Geison stain.

The severity of neointimal formation was scored on elastin-van Geison (EVG)-stained lung sections: the absence of neointimal formation equals 0; the presence of neointimal proliferation causing less than 50% luminal narrowing equals 1; luminal narrowing greater than 50% equals 2. Immunohistochemistry was performed on formalin-fixed, paraffin-embedded lung sections using antibodies to α-smooth-muscle actin (Dako, 1:30 dilution, linked to alkaline phosphatase), and CD31, a monoclonal antibody with specificity for endothelial cells lining the vascular lumen (Dako, 1:40 dilution, detected using avidin-biotin-peroxidase method). Samples of right lung were immediately placed in 2.5% paraformaldehyde and routinely processed for transmission electron microscopy. Reverse-transcription PCR was used to amplify portions of the rat endothelial nitric oxide synthase gene (eNOS, GenBank U02534) and actin B (GenBank V01217) from rat lung. Primer sequences for eNOS were: 5′-ctgggctccagagcataccc-3′ (SEQ ID NO:1) and 5′-gcagccttggcatcttctcc-3′ (SEQ ID NO:2). Five micrograms of total lung RNA were reverse-transcribed and amplified by PCR as described in Aoki, Y., et al. (1997) Am. J. Physiol. 272:L272-284. Endothelial NOS protein (60 μg) was analyzed by Western immunoblotting as described in Zhao, G. et al. (2000) Am. J. Physiol. 279:L958-L966, using anti-eNOS primary antibody (Santa Cruz sc-8311).

Results: All rats in the four treatment groups survived for the duration of the study and were assessed using hemodynamic measurements followed by sacrifice on postoperative day 35.

During the 35-day study period, control rats increased in body weight by 18%. In contrast, rats in Group PMV, that develop severe hypertensive pulmonary vascular disease following pneumonectomy, monocrotaline-injection and vehicle treatment, decreased in body weight by 15%. Rats treated with simvastatin after pneumonectomy and monocrotaline-injection preserved their body weights. Five to seven weeks after the monocrotaline injection, the pulmonary vessels showed 90% occlusion by a neointimal proliferation consisting of myofibroblasts, and the mean pulmonary artery pressures rise from a normal of 18 mmHg to over 45 mmHg. The right ventricle undergoes hypertrophy and the right ventricle/(left ventricle+septum) (RV/LV&S) ratio increases from 0.28 in normal rats to greater than 0.8 in rats with severe pulmonary arterial hypertension. Death from right ventricular congestive heart failure ensues within 1 to 3 weeks after pulmonary pressures surpass 40 mmHg.

Rats treated with simvastatin showed no significant differences in serum cholesterol at Day 35 compared to normal rats.

### Example 2

### Simvastatin Prevents the Development of Pulmonary Arterial Hypertension

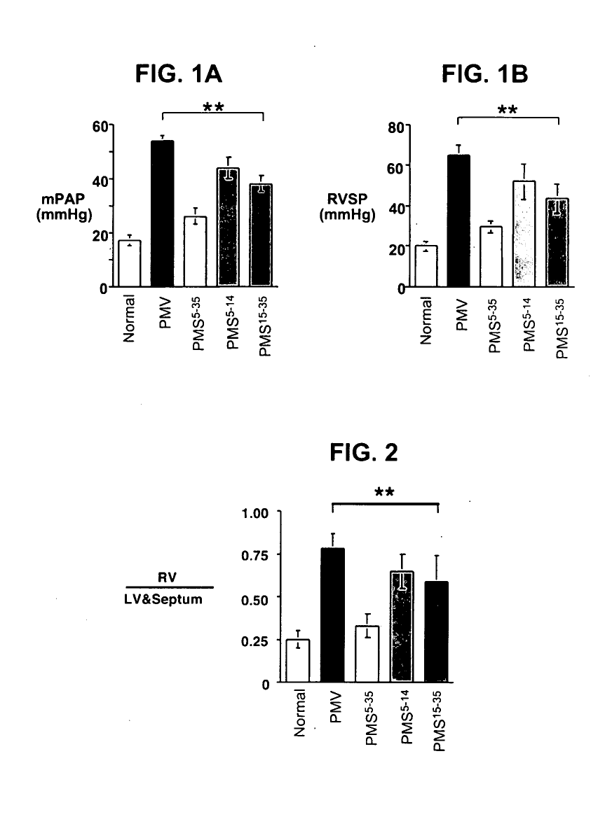

Hemodynamic measurements were performed as described in Example 1. Control rats (“Normal”) exhibited mean arterial blood pressures (126±9 mm Hg) and mean pulmonary arterial blood pressures (mPAP=17±1 mm Hg) that were typical of healthy adult rats. Pneumonectomized, monocrotaline-injected rats showed dramatically elevated mPAP and RVSP levels in comparison with control animals. In contrast, pneumonectomized, monocrotaline-injected rats treated with simvastatin showed no significant difference in systemic arterial blood pressure compared with normal rats. However, the simvastatin treated rats had elevated mean pulmonary arterial blood pressures, although they exhibited lower mPAP and RVSP values than rats that received only vehicle, as shown in FIGS. 1A and B.

Specifically, rats in Group PMS₅₋₃₅ exhibited the lowest mPAP (27±3 mm Hg) and RVSP (37±4 mm Hg) values. Rats in Group PMS₁₅₋₃₅ exhibited lower MPAP (38±3 mm Hg) and RSVP (54±10 mm Hg) values than those in Group PMS₅₋₁₄ (exhibiting mPAP values of 44±4 mm Hg, and RVSP values of 66±12 mm Hg). Of the four groups, rats in Group PMV, which received vehicle only, exhibited the highest mPAP (53±2 mm Hg) and RVSP (81±6 mm Hg) values. Two-way ANOVA and multiple comparison revealed that significant effects of simvastatin treatment on PAP occurred independently in the early simvastatin treatment group (Group PMS₅₋₁₄, p<0.001), and in the late simvastatin treatment group (Group PMS₁₅₋₃₅, p<0.001).

At Day 35, rats in control group PMV developed severe pulmonary arterial hypertension (mean PAP=53 mmHg), severe right ventricular hypertrophy (RV/LV&S=0.78), and severe vascular occlusion (vascular occlusion score=1.95). Rats treated continuously with simvastatin in group PMS5-35 showed substantial protection against the development of pulmonary hypertension (mean PAP=27 mm), protection against right ventricular hypertrophy (RV/LV&S=0.34) and protection against development of vascular occlusion (vascular occlusion score=1.5). Rats in the early and late simvastatin-treatment groups showed partial protection against the development of pulmonary hypertension.

### Example 3

### Simvastatin Prevents the Development of Right Ventricular Hypertrophy

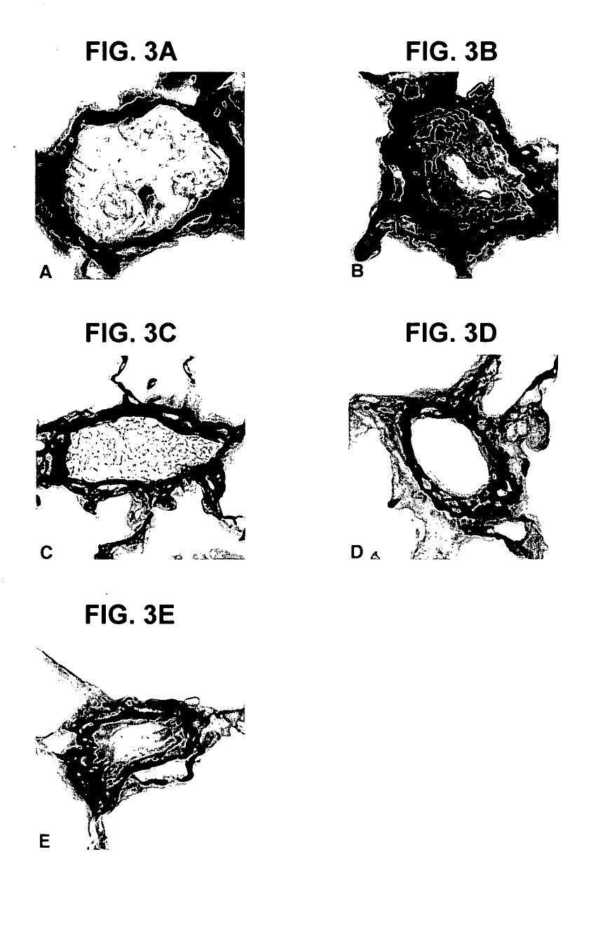

The development of chronic pulmonary arterial hypertension resulted in a compensatory hypertrophy of the right ventricle (increased ratio of (right ventricle)/(left ventricle and septum)(RV/LV+S)). The (RV/LV+S) ratio in normal rats is 0.25±0.03. Rats treated with simvastatin had lower (RV/LV+S) than rats that received vehicle (Group PMV=0.78±0.09), as shown in FIG. 2. The mean (RV/LV+S) ratios for the simvastatin-treated animals were: Group PMS₅₋₃₅=0.34±0.07; Group PMS₅₋₁₄=0.66±0.11; and Group PMS₁₅₋₃₅=0.60±0.15. Two-way ANOVA and multiple comparison revealed that the main effect occurred in the late simvastatin treatment group (Group PMS₁₅₋₃₅, p<0.01).

### Example 4

### Simvastatin Attenuates Pulmonary Artery Neointimal Formation

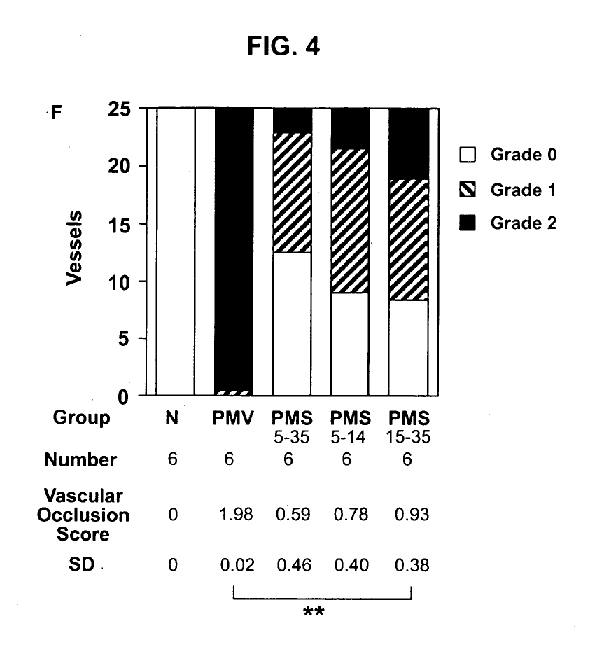

Representative morphologies of small pulmonary arteries from normal rats, and pneumonectomized, monocrotaline-injected rats, treated with vehicle or simvastatin, are shown in FIGS. 3A-3E. The tissues were stained for elastin to reveal the inner elastic lamina. FIG. 3A shows normal rat intra-acinar artery (Grade 0), with minimal EVG staining, at original magnification×600); FIG. 3B, by contrast shows a Grade 2 neointimal lesion (>50% narrowing) in pneumonectomized rats, four weeks after receiving monocrotaline (60 mg/kg s.c.), in the absence of simvastatin. The EVG staining indicates that extensive vascular remodeling had occurred, and demonstrates that the intimal lesions were causing obstruction. FIG. 3C shows a Grade 1 lesion demonstrating minimal intimal thickening in Group PMS₅₋₃₅ (Whole treatment group). FIG. 3D shows a Grade 1 lesion in Group PMS₅₋₁₄ (Early treatment group), while FIG. 3E shows a Grade 1 lesion in Group PMS₁₅₋₃₅ (Late treatment group).

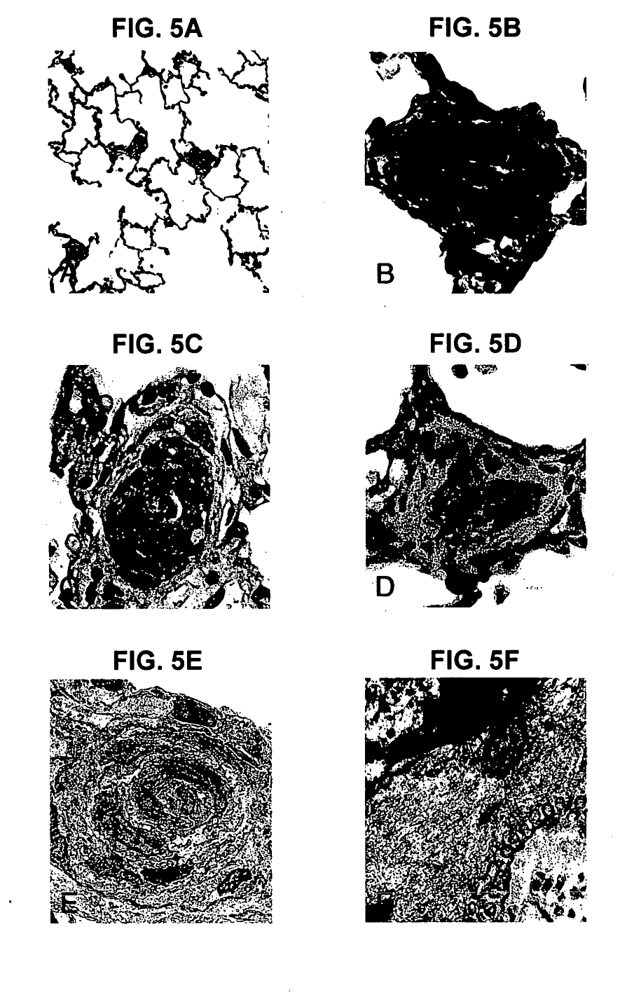

A quantitative analysis of luminal obstruction on 25 consecutive small pulmonary arteries from each rat in Group N, PMV, PMS₅₋₃₅, PMS₅₋₁₄ and PMS₁₅₋₃₅ was performed. The distribution of the vascular lesions, and an average vascular occlusion score (VOS) between 0 and 2 as defined above, are presented in FIG. 4. As shown in FIG. 4, normal rats show no neointimal formation, while rats in Group PMV develop severe pulmonary vascular remodeling, with a VOS of 1.98±0.02 (see also FIG. 3B vs. 3A). In contrast, rats treated with simvastatin showed significant decreases in the VOS compared with vehicle-treated rats. In particular, rats in Group PMS₅₋₃₅ showed a VOS of 0.59±0.46; rats in Group PMS₅₋₁₄ showed a VOS of 0.78±0.40; and rats in Group PMS₁₅₋₃₅ showed a VOS of 0.93±0.38. Two-way ANOVA and multiple comparison revealed that significant effects of simvastatin treatment on VOS occurred in both the early simvastatin treatment group (Group PMS₅₋₁₄, p<0.001), and in the late simvastatin treatment group (Group PMS₁₅₋₃₅, p<0.001).

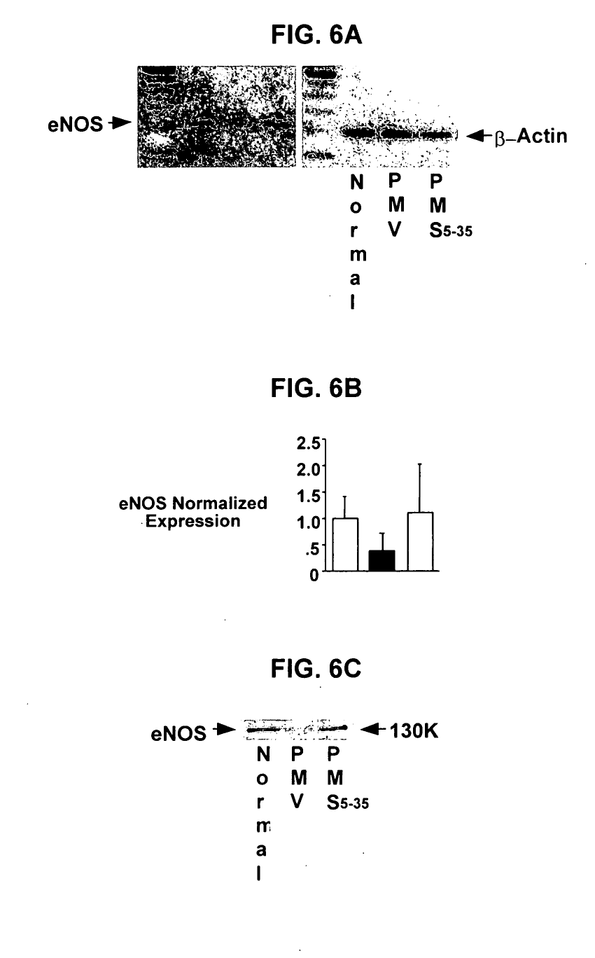

The cellular phenotype of the pulmonary microvascular neointimal occlusive lesions was also characterized. Hematoxylin and eosin-stained lung sections from Group PMV rats demonstrate extensive occlusion of small pulmonary arterioles, with preservation of alveolar structures (FIGS. 5A and B, 200× and 600× magnification, respectively), consisting of concentric laminar proliferations of spindled cells. As shown in FIG. 5C, most cells in a Grade 2 lesion stain positive (red) with immunohistochemical staining for α-smooth-muscle actin. The endothelial marker, CD31 monoclonal antibody, stains (brown) cells lining the vascular lumen (FIG. 5D). Electron microscopy was used to further characterize the neointimal lesions (FIGS. 5E and 5F, shown at 12,000× and 80,000× magnification, respectively). The electron micrographs show the differentiation of smooth muscle cells and the presence of disorganized myofibrils within the cells, but no fibronectin fibrils or fibronexus junctions, indicating myofibroblastic differentiation, were seen. The presence of α-smooth-muscle actin and disorganized myofibrils identifies these cells in the neointima to be smooth muscle cells.

### Example 5

### Endothelial Cell Nitric Oxide Synthase Levels were Restored Upon Treatment with Simvastatin

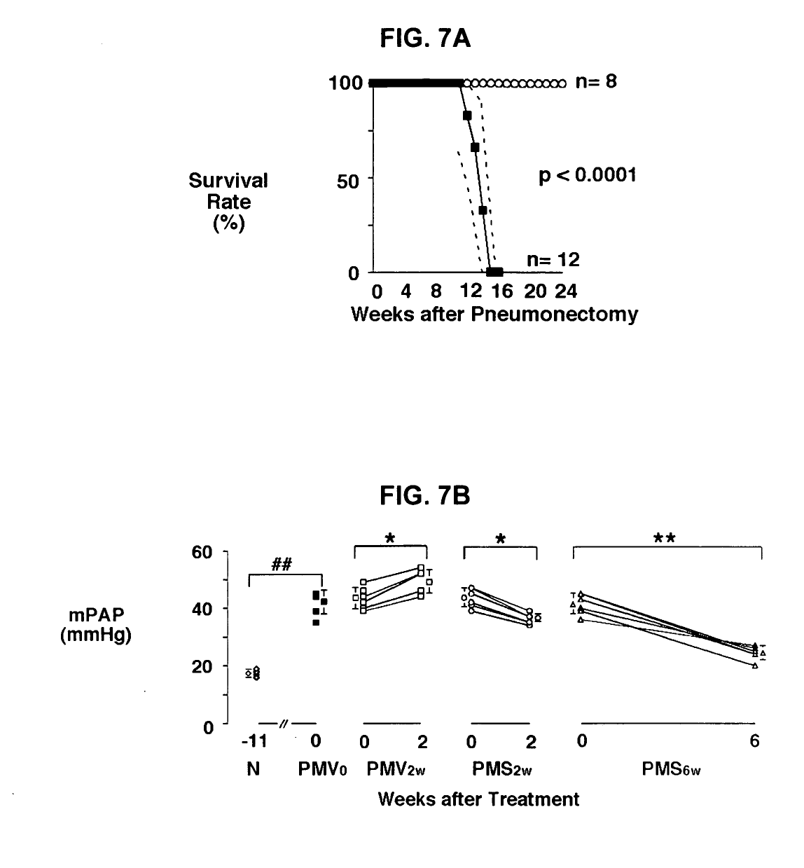

Changes in the lung eNOS mRNA expression were assessed semiquantitatively using reverse-transcription PCR (RT-PCR) (FIG. 6A). The expression of eNOS mRNA, normalized to α-actin expression, appeared to be significantly lower in diseased rats (Group PMV) than in normal rats (FIG. 6B). Simvastatin treatment increased expression of eNOS in pneumonectomized, monocrotaline-injected rats, although this trend did not achieve statistical significance. The lung expression of eNOS protein was assessed qualitatively by Western immunoblotting (FIG. 6C). An eNOS antisera-immunoreactive band is detected at 130 kDa. Compared to normal rats (Group N), diseased rats in Group PMV show decreased expression of eNOS protein, and simvastatin-treatment restored eNOS expression towards the level in normal rats.

These results show that eNOS expression is decreased during development of hypertensive pulmonary vascular disease and is restored by simvastatin. However, the change in expression of eNOS did not reach statistical significance. These results are consistent with the restoration of normal endothelial cell lining of the vasculature, and not with upregulation of eNOS.

### Example 6

### Pulmonary Arterial Hypertension in a Chronic Model and Rescue by Simvastatin

Methods: Rats were treated as described in Example 1, except that monocrotaline was administered four weeks after pneumonectomy. Pulmonary hypertension developed at Week 8 and increased in severity until death at Week 13. Rats were randomized to receive vehicle (Group PMV_{2w}) or simvastatin (2 mg/kg/day) by oral gavage beginning 7 weeks after administration of monocrotaline, when pulmonary hypertension had developed (Week 11). The rats to be administered simvastatin were divided into two groups for two weeks of treatment (Group PMS_{2w}) followed by sacrifice and study, or for six weeks of treatment (Group PMS_{6w}) followed by sacrifice and study. Hemodynamic measurements were performed on control and treated rats in order to determine mean arterial pressures and pressures at the right ventricle.

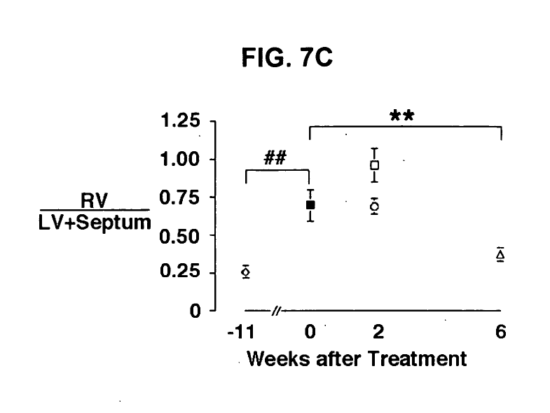

Results: The survival rates of the different treatment classes are shown in a Kaplan-Meier survival plot, depicted in FIG. 7A. Rats that received vehicle only for two weeks lost weight and scarcely moved prior to death in Week 15 (0% survival), FIG. 7A (filled squares). In contrast, all simvastatin-treated rats were spontaneously active, feeding and gaining weight from Week 11 onward and there was no mortality at Week 15, nor was there any mortality up to Week 23, when the animals were sacrificed for study (FIG. 7A, open circles). Thus, simvastatin conferred a 100% survival advantage compared to rats that received vehicle (p<0.001).

As shown in FIG. 7B, hemodynamic measurements from control rats demonstrated mean pulmonary arterial blood pressures (mPAP=17±1 mm Hg) that were typical of healthy adult rats (N, open diamonds). Pneumonectomized, monocrotaline-treated rats consistently developed severe pulmonary arterial hypertension by 11 weeks after pneumonectomy (MPAP=42±2 mmHg, RVSP=59±4 mmHg) (FIG. 7B, filled squares) at the time of randomization to vehicle or simvastatin treatment. Seventeen percent of rats randomized to receive vehicle, Group PMS_{2w}, died in Week 13, and the remainder were sacrificed for hemodynamic measurements that demonstrated progression of severe pulmonary arterial hypertension (MPAP=53±2 mm Hg, RVSP=72±5 mmHg) (FIG. 7B, open squares). The remaining rats that received vehicle all died of progression of pulmonary hypertension by 15 weeks.

In contrast, all rats rescued with simvastatin treatment survived and underwent scheduled measurements at 23 weeks. Rats treated with simvastatin for only two weeks, Group PMS_{2w}, showed MPAP values of 36±2 mm Hg and RVSP values of 53±7 mmHg (FIG. 7B, open circles), while rats treated for 6 weeks, Group PMS_{6w}, showed mPAP values of 24±3 mm Hg and RVSP values of 34±3 mmHg) (FIG. 7B, open triangles). The MPAP and RVSP values after 13 weeks of simvastatin treatment resemble those of normal rats (22±3 mm Hg, RVSP=30±3 mmHg, data not shown).

As shown in FIG. 7C, right ventricular hypertrophy was also decreased by simvastatin treatment relative to untreated rats, with a progressive decrease over time, nearly to levels observed in control rats (Group PMS_{2w}, open circle, and Group PMS_{6w}, open triangles, compared with normal rats, open diamond, and diseased rats prior to simvastatin treatment, filled square, and after two weeks treatment with vehicle only, open square).

Rats treated with simvastatin for 2 weeks and six weeks showed a progressive decrease in MPAP, RVSP and right ventricular hypertrophy toward levels observed in normal animals. These data support the conclusion that simvastatin treatment results in a reversal of established pulmonary arterial hypertension, and normalizes the mean pulmonary arterial pressures.

### Example 7

### Simvastatin Reverses Neointimal Vascular Occlusion

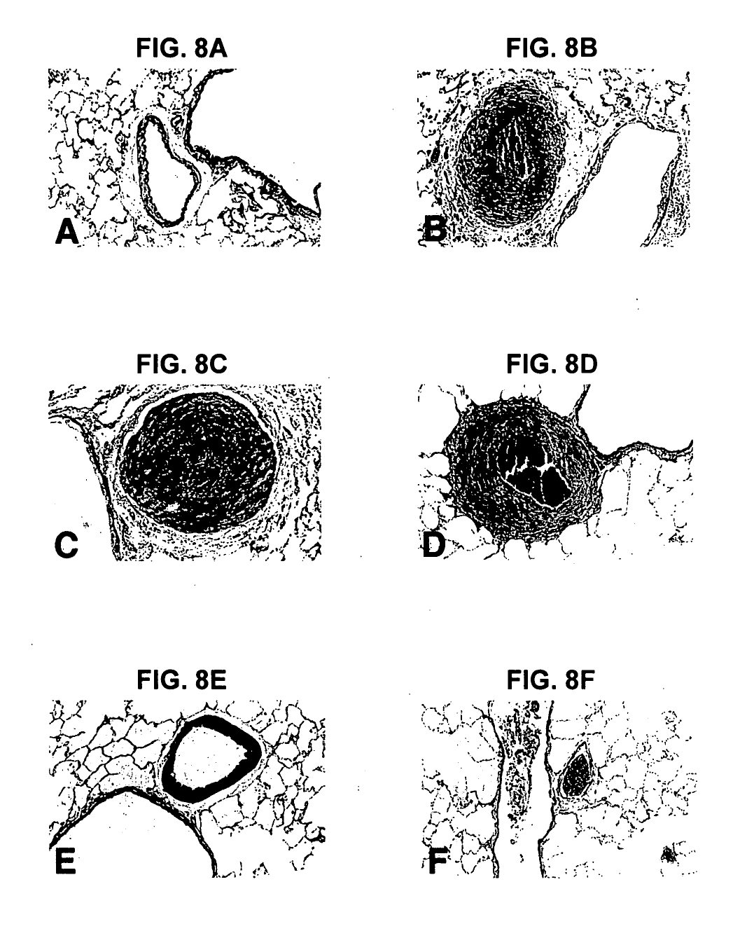

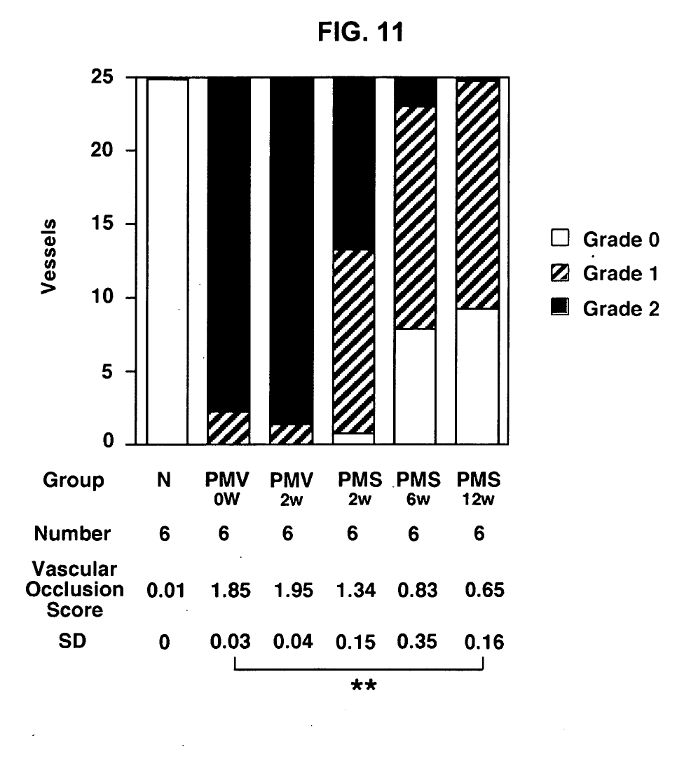

The administration of simvastatin is associated with decreased medial hypertrophy and neointimal occlusion and increased apoptosis of vascular smooth muscle cells. Eleven weeks after pneumonectomy, prominent medial wall hypertrophy and neointimal formation were evident in muscular pulmonary arteries on hematoxylin and eosin-stained lung sections from diseased rats, as shown in FIGS. 8A-8F. For comparison purposes, FIG. 8A shows a cross section of a normal peribronchial muscular pulmonary artery in a normal, untreated rat. In contrast, in pneumonectomized rats, after monocrotaline treatment (at 11 weeks, Group PMV_{0w}), a thickened medial layer in peribronchial muscular pulmonary artery is observed (FIG. 8B), exhibiting a high grade of vascular occlusion (see FIG. 11, discussed below). Rats that received vehicle for two weeks (Group PMV_{2w}) showed greater vascular obliteration with progression of medial hypertrophy and neointimal formation. In FIG. 8C, a prominent thickened medial layer and neointimal lesion can be seen. Intimal injury with endothelialitis was noted in many of the affected arteries. The thickened medial layer displayed smooth muscle proliferation admixed with pyknotic cells and acute inflammatory cells. The perivascular tissue spaces contained increased numbers of inflammatory cells embedded in a granulation tissue matrix. Plexiform lesions within muscular pulmonary arteries were not observed.

In contrast, rats treated with simvastatin for two weeks, (Group PMS_{2w}) demonstrated decreased medial hypertrophy (FIG. 8D), while rats treated with simvastatin for 6 weeks (Group PMS_{6w}) showed further improvements in luminal diameter (FIG. 8E). Rats treated with simvastatin for 13 weeks (Group PMS_{13w}) showed a complete regression of medial wall thickening, a resolution of the inflammation and near complete patency of the pulmonary arteries, likely the result of regression of medial hypertrophy and neointimal formation, as shown in FIG. 8F. All photographs were prepared with Hematoxylin and Eosin stain, at 40× magnification.

### Example 8

### Simvastatin Attenuated Proliferation and Induced Apoptosis of Pulmonary Artery Smooth Muscle Cells

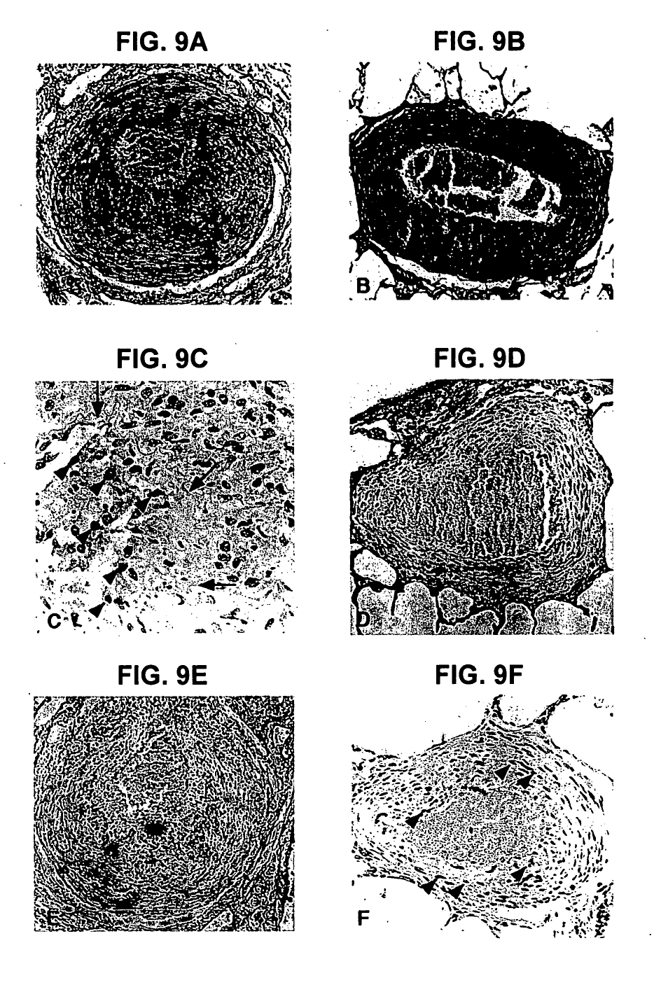

Rats that received vehicle (Group PMV_{2w}), showed prominent neointimal formation within muscular arteries (FIG. 9A). FIGS. 9A, 9C and 9E demonstrate representative histological results obtained from pneumonectomized, monocrotaline treated rats that received vehicle for 2 weeks (Group PMV_{2w}), while FIGS. 9B, 9D and 9F demonstrate representative histological results from pneumonectomized, monocrotaline treated rats that received simvastatin for two weeks (Group PMS_{2w}).

FIGS. 9A and B show EVG staining of peribronchial muscular pulmonary artery (×40) in rats in Groups PMV_{2w} and PMS_{2w}, respectively. Rats in Group PMV_{2w}, that received only vehicle, showed prominent neointimal formation within muscular arteries (FIG. 9A). In contrast, rats in Group PMS_{2w}, which were treated with simvastatin, showed essentially no neointima within the internal elastic lamina (FIG. 9B). Arrows show the internal elastic lamina, marking the inner diameter of the medial wall of the vessel. Normal vessels have a single layer of endothelial cells forming the intima within the internal elastic lamina.

The proliferation of vascular smooth muscle cells was characterized in situ using proliferating cell nuclear antigen (PCNA) immunochemical staining, as shown in FIGS. 9C and 9D (100× and 40×, respectively). Brown-staining nuclei are PCNA-positive cells that are prominent in neointimal lesions in Group PMV_{2w}, but are rare in Group PMS_{2w}. The proliferating cells are indicated with arrowheads in FIG. 9C. Arrows with stems indicate the internal elastic lamina. Large, peribronchial muscular pulmonary arteries from pulmonary hypertensive rats that received vehicle (Group PMV_{2w}) demonstrated prominent PCNA positive-staining cells in the neointima and medial wall (FIG. 9C). In contrast, pulmonary hypertensive rats treated with simvastatin for 2 weeks (Group PMS_{2w}) showed only rare PCNA positive-staining cells (FIG. 9D). These results demonstrate that simvastatin has an antiproliferative effect on vascular smooth muscle cells in pathological lesions within pulmonary arteries.

The regression of medial hypertrophy, neointimal formation and pulmonary hypertension achieved with simvastatin was likely to involve apoptosis of vascular lining cells. Apoptosis was analyzed in formalin-fixed lung sections by TUNEL assays (terminal deoxynucleotidyl transferase-mediated dUTP-biotin nick end-labeling) using the TACS™ 2 TdT Blue Label In situ Apoptosis detection kit (Trevigen, Gaithersberg, Md.). Proliferation was analyzed by using a monoclonal antibody against proliferating cell nuclear antigen (PCNA, 1:100 dilution, DAKO, Denmark), followed by biotin-SP-conjugated AffiniPure donkey anti-mouse IgG (1:400 dilution, Jackson ImmunoResearch, West Grove, Pa.), and then peroxidase-conjugated streptavidin (1:400 dilution, Jackson ImmunoResearch). TUNEL staining (blue) revealed increased numbers of TUNEL-positive nuclei in the medial walls and neointima of pulmonary arteries of rats treated with simvastatin for 2 weeks (Group PMS_{2w}) (FIG. 9F), compared to rats that received vehicle for two weeks (Group PMV_{2w}) (FIG. 9E). TUNEL-positive cells are indicated with arrowheads. No significant TUNEL-staining in smooth muscle cells of airways was observed.

Thus, in addition to an anti-proliferative effect, simvastatin treatment also promoted apoptosis of vascular smooth muscle cells in this model of pulmonary arterial hypertension.

### Example 9

### Simvastatin Reverses Neointimal Vascular Occlusion

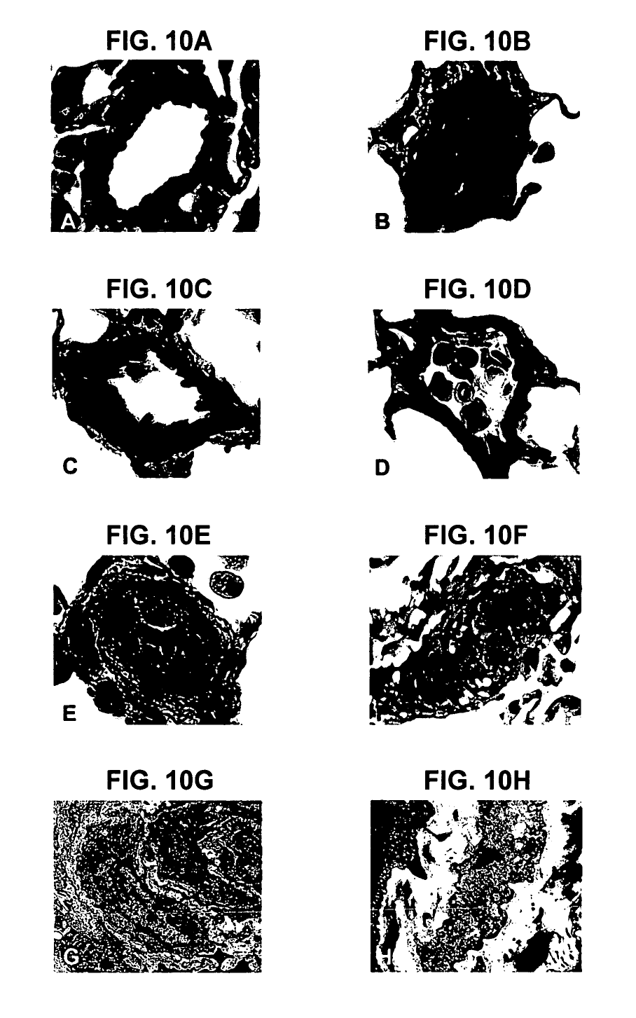

A quantitative analysis of neointimal luminal obstruction on 25 consecutive small pulmonary arteries from each rat in Group N, PMV_{0w}, PMV_{2w}, PMS_{2w}, PMS_{6w}, and PMS_{13w} was performed (FIGS. 10A-10D). Histological samples were prepared and stained using Elastin-van-Geison (EVG) stain, ×600. Vessels were assessed for neointimal proliferative lesions and scored: Grade 0 for no evidence of neointimal formation, Grade 1 for less than 50% luminal occlusion, Grade 2 for >50% luminal occlusion. The distribution of the vascular lesions, and an average vascular occlusion score (VOS) between 0 and 2, are presented in FIG. 11.

FIG. 10A shows a normal rat intra-acinar artery without evidence of neointimal proliferation (Grade 0), while FIG. 10B presents a Grade 2 neointimal lesion (>50% luminal occlusion) observed in a rat from Group PMV_{2w}. Simvastatin treated rats showed a regression in the degree of occlusion observed, with an improvement to a Grade 1 lesion (<50% luminal occlusion) after only two weeks of treatment with simvastatin (Group PMS_{2w}, FIG. 10C), and to a Grade 0 lesion after six weeks of simvastatin treatment (Group PMS_{6w}, FIG. 10D).

The interacinar arterial proliferation in Grade 2 lesion consists of spindled mesenchymal cells (FIG. 10E: H&E stain, ×600), and demonstrates immunoreactivity for α-smooth muscle actin (FIG. 10F: ×600). Electron microscopy of Grade 2 lesion demonstrates concentric proliferation of cells around a narrowed vascular lumen (FIG. 10G, TEM×6600). High power magnification shows alternate plaques and caveolae, indicative of smooth muscle differentiation (FIG. 10H, TEM×53,000).

The “vascular occlusion score” (VOS) is the average score for 25 consecutive intra-acinar pulmonary arteries. A VOS score of 0.01 was observed for arteries from a normal rat. A VOS score of 1.85 was observed for a sample from a rat at 11 weeks (Group PMV_{0w}), which increased to 1.95 in rats that received vehicle for 2 weeks (Group PMV_{2w}) (FIG. 11). Just two weeks of simvastatin treatment decreased the VOS to 1.34 (Group PMS_{2w}, and 6 weeks of treatment further decreased the VOS to 0.83 (Group PMS_{6w}). At 13 weeks of treatment, the VOS was 0.65 (Group PMS_{13w}) indicating near complete reversal of the neointimal vascular occlusion by simvastatin treatment. The extent of reversal of vascular occlusion greatly exceeded the increase that could be expected using a vasodilator. These results achieved statistical significance (**=p<0.01 by ANOVA).

### Example 10

### Simvastatin Downregulates Expression of Inflammatory Genes fos, jun, IL-1beta and TNF-alpha

Radiolabeled first-strand cDNA probes were generated from polyA+ RNA from 50 μg of total RNA extracted from homogenized whole lung, then hybridized to nylon arrays containing 1176 genes (Atlas Rat 1.2 Array Model 7854-1, Clontech Laboratories, Palo Alto, Calif.). After 4 high stringency washes, specific binding was detected by Southern hybridization (final wash with 0.1×SSC, 0.5% SDS at 68° C.). Signals were acquired and quantified using a Cyclone phosphorimager and Optiquant 3.0 software (Packard Instruments, Meriden, Conn.). The experiments were repeated at each time point with a total of three replicate animals. Using the Microsoft Excel spreadsheet program, the expression values for each gene at each time point were median-centered, averaged and log-transformed. The average of replicates for each time point was processed by average-linkage hierarchical clustering and k-means analysis using the J-Express software package (Bjarte Dysvik, University of Bergen, Norway).

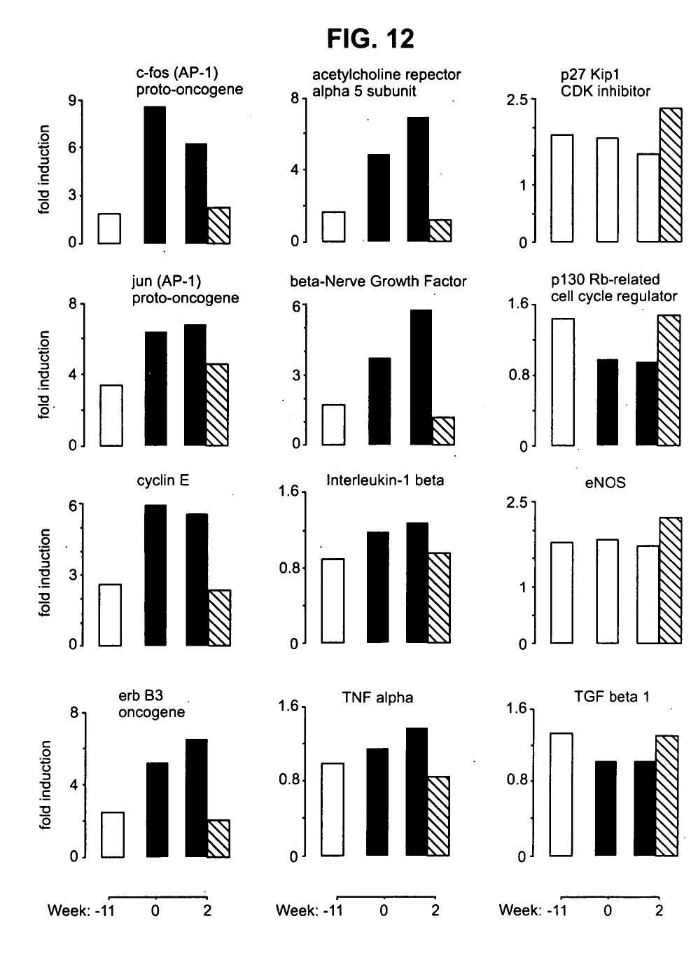

Selected genes from the ATLAS array were observed to show divergent expression between rats in control Group PMV_{2w} (vehicle treated rats, black bars) compared to the simvastatin-treated Group PMS_{2w} (grey bars). These results are graphically depicted in FIG. 12. The starting point is also shown, indicated as “0”, indicating the initial measurement, and both vehicle and simvastatin results are shown at the two week treatment period (“2”). Expression levels in normal lungs (“−11”, open bars) are shown for comparison.

The results of microarray analysis demonstrated increased expression of inflammatory genes, and inflammatory transcriptional regulators of the AP-1 family, c-fos and jun, and cytokines IL-1β and TNF-α in diseased tissues (FIG. 12). The administration of simvastatin inhibited AP-1 gene expression and suppression of AP-1 regulated inflammatory cytokines. Values represent averages of median-centered replicates for any given time point, so that an expression level of one represents the average of median gene intensities of the replicates for the given experimental time point.

These data are consistent with simvastatin providing an antiproliferative effect on vascular smooth muscle cells.

### Example 11

### Simvastatin Upregulates Expression of the Antiproliferative Regulators Cell Cycle Inhibitor p27Kip1 and p130 Rb-Related Protein

Gene expression profiles were generated as described in Example 10. Genes with diminished expression in pulmonary vascular disease that are restored or induced by simvastatin rescue treatment include antiproliferative regulators p27Kip1 and p130 Rb-related protein, as well as eNOS (FIG. 12, right column). Values represent averages of median-centered replicates for any given time point, so that an expression level of one represents the average of median gene intensities of the replicates for the given experimental time point.

These data are consistent with simvastatin providing an antiproliferative effect on vascular smooth muscle cells.

### Example 12

### Simvastatin Relieves Symptoms of Primary Pulmonary Hypertension in Human Patients

An open label clinical trial was performed on 16 human patients suffering from primary pulmonary hypertension. Half of these patients received concurrent intravenous prostacyclin treatment.

Methods: Subjects were initially evaluated at doses of 20 mg/day simvastatin. When no adverse effects were observed, the dosage was increased to 40 or 80 mg/day (two doses of 40 mg/day). Subjects were evaluated by performing an electrocardiogram, doppler echocardiogram and/or a right heart catheterization to measure pulmonary artery pressures. The efficacy of treatment with placebo or simvastatin was assessed by following 6-minute walk performance, exercise oxygen saturation, and doppler-echocardiogram measurements of pulmonary pressures and right ventricular function at 0, 3 months and 6 months. Because primary pulmonary hypertension is a progressive disease, the rate of decline in patient performance was also assessed as a measure of the efficacy of treatment.

Results: Subjects receiving simvastatin showed a decrease in progression of symptoms of disease, and improved performance in distance covered in the 6-minute walk tests. The greatest benefit was observed in subjects receiving the maximum dosage of simvastatin, 80 mg/day. There were no significant adverse effects associated with the administration of simvastatin to this group of patients.

### Example 13

### Double-Blind Controlled Study of Simvastatin for Treatment of Pulmonary Hypertension

A double-blind controlled study is being performed at Stanford University in order to assess the efficacy of simvastatin in treating patients with pulmonary hypertension. Subjects receive simvastatin initially at 40 mg/day. The effects of simvastatin treatment at 40 mg/day are studied for 12 weeks.

Subjects are evaluated before and during treatment, which includes an electrocardiogram, a doppler echocardiogram and/or a right heart catheterization to measure pulmonary artery pressures. Clinic evaluations by physician-investigators, exercise performance measurements and blood sampling are performed at four-week intervals. Blood tests include complete blood count, serum electrolytes, renal and liver function tests, cholesterol, creatine phosphokinase, coagulation studies and thyroid function tests.

The efficacy of treatment with placebo or simvastatin is assessed by following 6-minute walk performance, exercise oxygen saturation, and doppler-echocardiogram measurements of pulmonary pressures and right ventricular function at 0, 6, and 12-weeks.

The study may demonstrate the efficacy of simvastatin in reversing vascular obstruction and/or decreasing the rate of progression of primary pulmonary hypertension.

1. A method of treating a lung proliferative vascular disorder in a patient comprising administering an HMG-CoA reductase inhibitor, wherein the HMG-CoA reductase inhibitor is present in an amount effective to reduce vascular occlusion in the pulmonary arteries of the patient, and which does not substantially increase endothelial cell nitric oxide synthase activity in the endothelial cells of the pulmonary arteries of the patient.

2. The method of claim 1, wherein the lung proliferative vascular disorder is selected from the group consisting of primary pulmonary hypertension, secondary pulmonary hypertension, Eisenmenger's syndrome, chronic thromboembolic disease, pulmonary fibrosis, obliterative bronchiolitis, and lymphangioleiomyomatosis.

3. The method of claim 2, wherein the lung proliferative vascular disorder is primary pulmonary hypertension.

4. The method of claim 1, wherein the HMG-CoA reductase inhibitor is selected from the group consisting of atorvastatin, cerivastatin, fluvastatin, lovastatin, mevastatin, pravastatin, pitastatin, rosuvastatin and simvastatin.

5. The method of claim 1, wherein the HMG-CoA reductase inhibitor is simvastatin.

6. The method of claim 1, wherein the HMG-CoA reductase inhibitor is administered in a pharmaceutical formulation at a dose of from about 0.1 to about 100 mg/kg per day.

7. The method of claim 6, wherein the formulation further comprises a pharmaceutically acceptable carrier suitable for oral, parenteral, transdermal, transmucosal, or pulmonary delivery.

8. The method of claim 1, further comprising administering an additional active agent.

9. The method of claim 8, wherein the additional active agent is selected from the group consisting of anticoagulants, vasodilators, macrolide anti-inflammatory agents, diterpenoid triepoxides, endothelin receptor antagonists, geranyl transferase inhibitors, farnesyl transferase inhibitors, and inhibitors of EGF tyrosine kinase, and pharmaceutically acceptable salts and esters thereof.

10. The method of claim 9, wherein the additional active agent is a vasodilator selected from the group consisting of prostanoids, phosphodiesterase (PDE) inhibitors, nitric oxide, nitric oxide precursors and calcium channel blockers.

11. The method of claim 10, wherein the calcium channel blocker is nifedipine or diltiazem.

12. The method of 10, wherein the prostanoid is prostacylin, treprostinil, iloprost, beraprost, prostaglandin E₁ or prostaglandin E₂.

13. The method of claim 12, wherein the prostanoid is prostacyclin.

14. The method of claim 9, wherein the vasodilator is a PDE inhibitor.

15. The method of claim 14, wherein the PDE inhibitor is a PDE (V) inhibitor.

16. The method of claim 9, wherein the macrolide anti-inflammatory agent is rapamycin and its derivatives, FK506, erythromycin or azithromycin.

17. The method of claim 9, wherein the diterpenoid triepoxide is triptolide.

18. The method of claim 9, wherein the endothelin receptor antagonist is ambrisentan, BMS207940, bosentan, sitaxsentan or tezosentan.

19. The method of claim 1, wherein neointimal smooth muscle cell hyperplasia is decreased upon treatment with the antiproliferative agent, thereby reducing the neointimal smooth muscle cell hyperplasia in the pulmonary arteries of the patient.

20. The method of claim 1, wherein the lung proliferative vascular disorder is characterized by vascular occlusion in the pulmonary arteries of the patient, and wherein the vascular occlusion is reversed upon treatment with the antiproliferative agent, such that an increase in blood flow is provided through the pulmonary arteries.

21. The method of claim 20, wherein the blood flow is increased by from about 5% to at least about 300%.

22. The method of claim 1, wherein the lung proliferative vascular disorder is characterized by pulmonary hypertension, and wherein the hypertension is reversed upon treatment with the antiproliferative agent.

23. The method of claim 7, wherein the antiproliferative agent is administered by inhalation.

24. The method of claim 23, wherein the antiproliferative agent is administered using a dry powder inhaler, metered dose inhaler, or nebulizer.

25. A method of preventing vascular occlusion in a patient predisposed to developing pulmonary hypertension, comprising administering an antiproliferative agent to the patient.

26. The method of claim 25, wherein the patient is predisposed to develop pulmonary hypertension due to a genetic defect in the TGF-β superfamily of receptors of the patient.

27. The method of claim 26, wherein the genetic defect is a haploinsufficiency of bone morphogenetic protein receptor II (BMPR2).

28. The method of claim 27, wherein the antiproliferative agent is an HMG-CoA reductase inhibitor.

29. The method of claim 28, further comprising administering an additional active agent selected from the group consisting of anticoagulants, vasodilators, diterpenoid triepoxides, and endothelin receptor antagonists, macrolide anti-inflammatory agents, geranyl transferase inhibitors, farnesyl transferase inhibitors, and inhibitors of EGF tyrosine kinase receptor signaling and pharmaceutically acceptable salts and esters thereof.

30. A method of reversing right ventricular hypertrophy in a patient suffering from pulmonary hypertension comprising administering an antiproliferative agent.

31. The method of claim 30, wherein the antiproliferative agent is an HMG-CoA reductase inhibitor.

32. The method of claim 31, further comprising administering an additional active agent selected from the group consisting of anticoagulants, vasodilators, macrolide anti-inflammatory agents, diterpenoid triepoxides, inhibitors of EGF tyrosine kinase receptor signaling, geranyl transferase inhibitors, farnesyl transferase inhibitors, and endothelin receptor antagonists, and pharmaceutically acceptable salts and esters thereof.

33. A method of treating pulmonary hypertension comprising administering an antiproliferative agent.

34. The method of claim 33, wherein the antiproliferative agent is an HMG-CoA reductase inhibitor.

35. The method of claim 34, wherein the HMG-CoA reductase inhibitor is simvastatin.

36. The method of claim 35, wherein the method of administration is selected from the group consisting of pulmonary, oral, transmucosal, transdermal and parenteral administration.

37. The method of claim 36, wherein the HMG-CoA reductase inhibitor is administered by inhalation.

38. The method of claim 34, wherein the HMG-CoA reductase inhibitor is administered in a dose of from about 0.1 to about 100 mg/kg per day.

39. The method of claim 34, wherein the formulation further comprises an additional active agent selected from the group consisting of anticoagulants, vasodilators, macrolide anti-inflammatory agents, FK506, inhibitors of EGF tyrosine kinase receptor signaling, diterpenoid triepoxides, geranyl transferase inhibitors, farnesyl transferase inhibitors, and endothelin receptor antagonists, and pharmaceutically acceptable salts and esters thereof.
